(19) United States
(12) Patent Application Publication
(10) Pub. No.: US 2023/0241450 A1
(43) Pub. Date: Aug. 3, 2023

(54) WORKOUT MAT

(71) Applicant: Lululemon Athletica Canada Inc., Vancouver (CA)

(72) Inventors: Daljinder Singh SANGHERA, Vancouver (CA); Navjot KAILAY, Vancouver (CA); William LY, Vancouver (CA); Joseph John SANTRY, Vancouver (CA); Ellisa Kathleen CALDER, Vancouver (CA); Kerem DOGURGA, Vancouver (CA); Nils Johan EDZÉN, Vancouver (CA); Erica Margaret BUCKERIDGE, Vancouver (CA)

(21) Appl. No.: 17/984,070

(22) Filed: Nov. 9, 2022

Related U.S. Application Data
(60) Provisional application No. 63/278,940, filed on Nov. 12, 2021.

(51) Int. Cl. A63B 21/00; A63B 24/00; A63B 71/06

(52) U.S. Cl. CPC A63B 21/4037; A63B 24/0062; A63B 71/0619; A63B 2024/0093; A63B 2071/0675; A63B 2220/833

(57) ABSTRACT

Embodiments described herein relate to systems and methods for a connected workout mat to create sensory experiences. A connected mat has at least one input device to receive input data and at least one output device that synchronises with content through a digital content platform. A hardware processor generates digital output for the digital content platform or physical sensory output at the connected mat based on the input data from the connected mat and instructor input. Embodiments described herein relate to systems and methods for a workout mat with at least one heating region to create sensory experiences based on input data.

### FIELD

The improvements generally relate to the field of computers, exercise, workout mats, immersive hardware.

### INTRODUCTION

Workout mats can be used for exercise to provide a durable and comfortable surface. Workout mats can be used for different types of exercises such as yoga, stretching, weight training, interval training, and dance, for example.

### SUMMARY

In an aspect, embodiments described herein provide systems and methods for a connected workout mat, a processor and a digital content platform. In an aspect, embodiments described herein provide a workout mat.

In an aspect, embodiments described herein provide a workout mat to enhance digital connection through sensory output of a physical connected workout surface of the mat responsive to input, wherein the workout mat comprises at least one input control to receive a selected pattern of mat output from a plurality of patterns of mat output, and at least one output component to provide sensory output according to the selected pattern of mat output.

In some embodiments, the plurality of patterns of mat output comprise at least one selectable pre-programmed pattern of mat output.

In some embodiments, the plurality of patterns of mat output comprise at least one modifiable pattern of mat output.

In some embodiments, a client application on an electronic device has a user interface to generate a user profile to define one or more attributes of the selected pattern of mat output.

In some embodiments, the one or more attributes comprise a combination of activity and duration.

In some embodiments, the one or more attributes are embedded mat outputs linked to at least one of a selected exercise and a selected experience.

In some embodiments, the mat is connectable to at least one input device to receive input data to trigger content at a digital content platform, the content comprising at least one of pre-programmed content and user specified content.

In some embodiments, the input control receives instructions for the sensory output from exercise content, the instructions being embedded within the exercise content.

In some embodiments, the input control receives instructor input from an instructor device to modify the instructions for the sensory output.

In some embodiments, the at least one output component synchronises the sensory output with content at a digital content platform.

In some embodiments, the sensory output of the physical connected workout surface is controlled or activated by exercise content of a digital content platform, wherein the content has one or more timestamps or metadata to synchronize the sensory output with the exercise content.

In some embodiments, the mat has a plurality of layers comprising a non-slip textile surface layer, a multi-zone heating layer, a sensor layer, a mat layer.

In some embodiments, the input control comprises a low-power controller integrated into the mat to consolidate signals from sensors of the sensor layer, and direct controls to zones of the multi-zone heating layer, wherein power and data is transmitted to the controller by a connector.

In some embodiments, the low-power controller is removable.

In some embodiments, the mat has a light portion that provides an indicator and a touch surface.

In some embodiments, the input control comprises an input surface and a sensor layer integrating a plurality of sensors.

In some embodiments, the at least one input control captures interactions with the connected mat to trigger control commands relating to the selected pattern of pattern of mat output, the interactions comprising gesture recognition, taps in zones on the mat, and intelligent activity recognition based on context.

In another aspect, embodiments described herein provide a system for a workout mat to create sensory experiences. The system has a workout mat with at least one input device to receive input data for a pattern of map output and at least one output component to provide a sensory output according to the pattern of map output. The system has a hardware processor that receives selected exercise content of a digital content platform and provides the pattern of map output to the at least one input control based on the selected exercise content to synchronize the selected exercise content of the digital content platform and the sensory output at the connected mat.

In some embodiments, the system has a client application on an electronic device with a user interface to generate a user profile to define one or more attributes of the pattern of mat output.

In some embodiments, the hardware processor processes the input data using a trained model.

In some embodiments, the hardware processor updates the trained model based on data captured from other users.

In some embodiments, the hardware processor trains the trained model based on user data.

In some embodiments, the digital content platform is an immersive hardware device.

In some embodiments, the hardware processor controls content by interactions with the connected mat captured by at least one input control to trigger control commands relating to the pattern of mat output, the interactions comprising gesture recognition, taps in zones on the mat, and intelligent activity recognition based on context.

In some embodiments, the system has an instructor device that provides control commands to the digital content platform to modify the pattern of map output.

In some embodiments, the output device is controlled by a remote instructor device for direct manipulation and control a workout experience through a range of outputs comprising heating, cooling, and vibration exhibited through the connected mat.

In another aspect, embodiments described herein provide a method for a connected workout mat to create sensory experiences. The method involves: receiving a selected pattern of mat output from a connected mat having at least one input control; and generating sensory output by at least one output component of the connected mat based on the selected pattern of map output.

In some embodiments, the method involves providing a plurality of selectable programmed patterns of mat output to receive the selected pattern of mat output.

In some embodiments, the method involves receiving at least one modification for the selected pattern of mat output from a client application, modifying the selected pattern of mat output based on the modification, and generating the sensory output at the connected mat based on the modified selected pattern of map output.

In some embodiments, the method involves determining the selected pattern of map output by: signal and data filtering of input data by a hardware processor; and processing the input data by the hardware processor using a trained model.

In some embodiments, the method involves updating the trained model over time based at least on one dataset where the dataset relates to at least one of user specific data, household specific data, ambient environment data, community data, expert data, instructor data, user goal data, community goal data, instructor goal data.

In some embodiments, the method involves training the trained model with data from at least one of community data, expert data, instructor data.

In some embodiments, the method involves controlling the sensory output by interactions with the connected mat captured by the at least one input control, the interactions comprising gesture recognition, taps in zones on the mat, and intelligent activity recognition based on context.

In some embodiments, the method involves receiving control commands from an instructor device for the selected pattern of map output.

In some embodiments, the method involves providing exercise content from at least one of the digital content platform; and synchronizing the sensory output with the exercise content provided by the digital content platform.

In a further aspect, embodiments described herein provide a connected workout mat to create multisensory experiences, wherein the connected mat comprises at least one input control to receive a selected pattern of mat output from a plurality of patterns of mat output and at least one output component to provide sensory output based on the selected pattern of mat output and synchronizing the sensory output with content from a digital content platform, wherein the connected mat has a connection to a hardware processor that provides instructions for the selected pattern of mat output and the content.

In some embodiments, the hardware processor is removable from the mat.

In some embodiments, the hardware processor is embedded in the mat.

In some embodiments, the mat has a plurality of layers comprising a non-slip textile surface layer, a multi-zone heating layer, a sensor layer, a mat layer.

In some embodiments, the input control comprises a low-power controller integrated into the mat to consolidate signals from sensors of the sensor layer, and direct controls to zones of the multi-zone heating layer, wherein power and data is transmitted to the controller by a connector.

In some embodiments, the mat has a light portion that provides an indicator and a touch surface.

In some embodiments, the at least one input device comprises an input surface and a sensor layer integrating a plurality of sensors.

In some embodiments, the least one input control captures interactions with the connected mat, the interactions comprising gesture recognition, taps in zones on the mat, and intelligent activity recognition based on context.

In some embodiments, the input control receives instructor input by a remote instructor device for direct manipulation and control of the sensory output for a workout experience through a range of outputs comprising heating, cooling, and vibration exhibited through the connected mat.

In another aspect, embodiments described herein provide a workout mat to create sensory experiences, wherein the connected mat comprises at least one input control to receive input data for a pattern of mat output and at least one heating element, wherein the connected mat generates sensory output using the at least one heating element based on the selected pattern of mat output.

In another aspect, embodiments described herein provide a workout mat to create sensory experiences, wherein the connected mat comprises at least one input control to receive input data for a pattern of mat output and at least display, wherein the connected mat generates visual output using the display based on the input data.

In another aspect, embodiments described herein provide a system for providing a user with a sensory heat response to support an activity. The system has a workout mat comprising a heat region, at least one input control to receive input data for a pattern of map output, and at least one output component to control the heat in the workout mat. The system has non-transitory memory storing activity data for recommended heating for an activity. The system has a hardware processor programmed with executable instructions to receive information about the activity, determine heat models based on the activity and generate heat instructions for the heat region of the workout mat, wherein the output component controls the heat in the workout mat based on the heat instructions and the pattern of map output.

In some embodiments, the workout mat has a multi-zone heat region.

In some embodiments, the workout mat comprises non-slip textile surface layer, a multi-zone heating layer, a sensor layer, a mat layer.

In some embodiments, the hardware processor determines an ambient temperature of the location of the workout mat as an input to generating the heat instructions.

In some embodiments, an activity comprises a series of related activities.

In some embodiments, an activity is associated with an activity type.

In some embodiments, the heat instructions comprise at least one of heating a heat region to more than one temperature during the duration of the activity, cooling a heat region to more than one temperature during the duration of the activity.

In some embodiments, the executable instructions determine the activity from one or more activities of a connected digital platform input.

In some embodiments, the executable instructions determine the activity from a pre-recorded activity input.

In some embodiments, the executable instructions determine the activity from an audio input.

In some embodiments, the executable instructions determine the activity from stored information that comprises at least one of a standard series for an activity type, a repetitive series, a progressive series.

In some embodiments, the non-transitory memory and hardware processor are embedded within the exercise mat.

In some embodiments, the at least one input device comprises an input surface and a sensor layer integrating a plurality of sensors.

In some embodiments, the mat has a low-power controller integrated into the mat to consolidate signals from sensors of the sensor layer, and direct controls to the heat region, wherein power and data is transmitted to the controller by a connector.

In some embodiments, the at least one input control captures interactions with the workout mat, the interactions comprising gesture recognition, taps in zones on the mat, and intelligent activity recognition based on context.

In another aspect, there is provided a method for providing a user with a sensory heat response to support an activity. The method involves: receiving input data from at workout mat by at least one input control; receiving input data identifying an activity; generating heat instructions for a heat region of the workout mat based on the activity; and heating the heat region of the workout mat based on heat instructions.

In some embodiments, the method involves generating the heat instructions by processing the input data by a hardware processor using a trained model.

In some embodiments, the heat region comprises multiple heat-regions receiving heat instructions.

In some embodiments, an activity comprises a series of related activities.

In some embodiments, the method involves identifying an activity from stored information that comprises at least one of a standard series for an activity type, a repetitive series, or a progressive series.

In some embodiments, the method involves associating a time duration with the activity.

In some embodiments, the heat instructions involve code for heating the heat region to more than one temperature during the duration of the activity.

In some embodiments, the method involves generating heat instructions based on the time duration associated with the activity.

In some embodiments, at least one input control captures interactions with the workout mat, the interactions comprising gesture recognition, taps in zones on the mat, and intelligent activity recognition based on context.

In some embodiments, the activity is associated with an activity type.

In some embodiments, the input data identifying the activity is received from a connected digital platform.

In some embodiments, the input data identifying the activity is received from a pre-recorded activity.

In some embodiments, the input data identifying the activity is received from an audio input.

Embodiments described herein provide a system for a workout mat to create sensory experiences. The system has a workout mat with at least one input device to receive input data and at least one output device that synchronises with content through a digital content platform. The system has a hardware processor that generates digital output for the digital content platform or physical sensory output at the connected mat based on the input data from the connected mat and instructor input.

In some embodiments, the workout mat comprises non-slip textile surface layer, a heating layer, a sensor layer, a mat layer. In some embodiments the heating layer has one or more zones that generate heat. In some embodiments, the heating layer has multiple zones that generate heat, which may be referred to as a multi-zone heating layer.

In some embodiments, the workout mat comprises a low-power removable controller integrated into the mat to consolidate signals from sensors of the sensor layer, and direct controls to zones of the multi-zone heating layer. In some embodiments, power and data is transmitted to the controller by a connector.

In some embodiments, the workout mat comprises a light portion that provides an indicator and a touch surface.

In some embodiments, the at least one input device comprises an input surface and a sensor layer integrating a plurality of sensors.

In some embodiments, the hardware processor processes the input data using a pre-trained model.

In some embodiments, the digital content platform is an immersive hardware device.

In some embodiments, the hardware processor controls content by interactions with the connected mat captured by the at least one input device, the interactions comprising gesture recognition, taps in zones on the mat, and intelligent activity recognition based on context.

In some embodiments, the system has an instructor device that provides control commands to the digital content platform and received video of a user on the connected workout mat captured by digital content platform.

In some embodiments, the output device is controlled by a remote instructor device for direct manipulation and control a workout experience through a range of outputs comprising heating, cooling, and vibration exhibited through the connected mat.

Embodiments described herein provide a method for a connected workout mat to create sensory experiences. The method involves: receiving input data from a connected workout mat by at least one input device and user interface with the connected workout mat; signal and data filtering of the input data by a hardware processor; processing the input data by the hardware processor using a trained model; generating digital output for the digital content platform or physical sensory output for at least one output device of the connected mat based on the input data from the connected workout mat and instructor input; and synchronizing the physical sensory output with content through a digital content platform.

In some embodiments, the method involves controlling content by interactions with the connected workout mat captured by the at least one input device, the interactions comprising gesture recognition, taps in zones on the mat, and intelligent activity recognition based on context.

In some embodiments, the method involves receiving control commands from an instructor device.

Embodiments described herein provide a workout mat to create sensory experiences. The workout mat comprises at least one input device to receive input data and at least one output device that synchronises with content through a digital content platform. In some embodiments, the workout mat can have a non-slip textile surface layer, a heating layer, and a mat layer. In some embodiments, the workout mat can have a heating layer with multiple heating zones, or a multi-zone heating layer. The workout mat with the heating layer having multiple heating zones, has a controller to enable manual control and automated control to sync to a program of the digital content platform. In some embodiments, the workout mat has a sensor layer to capture sensor data.

In some embodiments, wherein the workout mat is a connected workout mat has a connection to a hardware processor that generates digital output for the digital content platform or physical sensory output at the connected mat based on the input data from the connected mat and instructor input.

In some embodiments, the connected workout mat has a low-power removable controller integrated into the mat to consolidate signals from sensors of the sensor layer, and direct controls to zones of the multi-zone heating layer, wherein power and data is transmitted to the controller by a connector.

In some embodiments, the connected workout mat has an embedded non-removable low-power controller integrated into the mat to consolidate signals from sensors of the sensor layer, and direct controls to zones of the multi-zone heating layer, wherein power and data is transmitted to the controller by a connector.

In some embodiments, the connected workout mat has a light portion that provides an indicator and a touch surface.

In some embodiments, the workout mat has a heating layer with one or more zones that generate heat. The workout mat with the heating layer can also have one or more input sensors in some example embodiments.

In some embodiments, the at least one input device comprises an input surface and a sensor layer integrating a plurality of sensors.

In some embodiments, the least one input device captures interactions with the connected mat captured by the at least one input device, the interactions comprising gesture recognition, taps in zones on the mat, and intelligent activity recognition based on context.

In some embodiments, the digital content platform includes pre-programmed content and/or user specified content.

In some embodiments, the output device is controlled by a remote instructor device for direct manipulation and control of a workout experience through a range of outputs comprising heating, cooling, and vibration exhibited through the connected mat.

In some embodiments, the system provides a sensory heat response to support an activity through the workout mat with at least one heat region and at least one input device to receive and one output device to control the heat in the workout mat.

In another aspect, there is provided a non-transitory memory that stores activity information records and recommended heating for an activity and a hardware processor programmed with executable instructions to receive information about the activity, determine heat models based on the activity and generate heat instructions for one or more heat regions or zones of the workout mat.

In some embodiments, the workout mat comprises a non-slip textile surface layer, a heating layer with one or more heat zones, a sensor layer, a mat layer. In some embodiments the heat layer is multi-zone.

Many further features and combinations thereof concerning embodiments described herein will appear to those skilled in the art following a reading of the instant disclosure.

### DESCRIPTION OF THE FIGURES

In the figures,

FIG. 1A shows an example embodiment of a workout mat.

FIG. 1B shows an example system for a connected workout mat according to embodiments described herein.

FIG. 1C shows an example system for a connected workout mat according to embodiments described herein.

FIG. 2 shows an exploded view of a connected workout mat according to embodiments described herein.

FIG. 3 shows another example system of peripherals for a connected workout mat according to embodiments described herein.

FIG. 4 shows a view of an example surface for a connected workout mat according to embodiments described herein.

FIG. 5 shows an example connector and light portion of a connected workout mat according to embodiments described herein.

FIG. 6 shows an example flow diagram of a method for a connected workout mat according to embodiments described herein.

FIG. 7 shows an example architecture diagram of a system for a connected workout mat according to embodiments described herein.

FIG. 8A shows an example diagram of a system for a connected workout mat according to embodiments described herein.

FIG. 8B shows an example diagram of a system for a connected workout mat according to embodiments described herein.

FIG. 8C shows an example diagram of a system for a connected workout mat according to embodiments described herein.

FIG. 9 shows an example architecture diagram for a workout mat according to embodiments described herein.

FIG. 10 shows an example flow system diagram for a workout mat according to embodiments described herein.

FIG. 11 shows an exploded view of a workout mat example flow system diagram for a workout mat according to embodiments described herein.

FIG. 12 shows an example architecture diagram for a workout mat according to embodiments described herein.

FIG. 13 shows an example user device according to embodiments described herein.

FIG. 14 shows another example illustration of system that can provide a sensory experience for exercise or activity content according to embodiments described herein.

FIG. 15 shows an example portion of a mat that is configured to provide visual output.

FIG. 16 shows an example mat with a mat antenna that can couple wirelessly to one or more peripherals.

FIG. 17 shows an example of different community immersive examples or gestures to connect with other users using the mat.

FIG. 18 is another example diagram of an exploded view of the mat to illustrate different layers of the mat.

FIG. 19 illustrates example heat experiences generated by mat.

FIG. 20 illustrates further example heat experiences generated by mat for engaging new content.

FIG. 21 illustrates further example heat experiences generated by mat for different exercises or activities.

FIG. 22 illustrates further example heat experiences generated by mat.

FIG. 23 illustrates example control experiences generated by mat.

FIG. 24 illustrates example experiences generated by mat.

FIG. 25 illustrates example experiences generated by mat for smart home integration.

### DETAILED DESCRIPTION

Embodiments described herein relate to a workout mat to provide sensory experiences, and systems and methods for the workout mat to provide the sensory experiences.

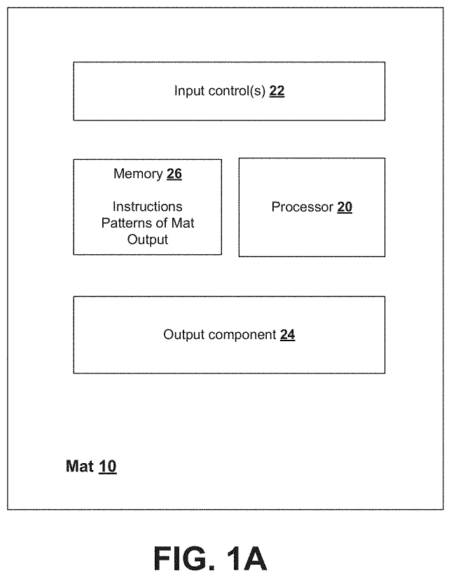

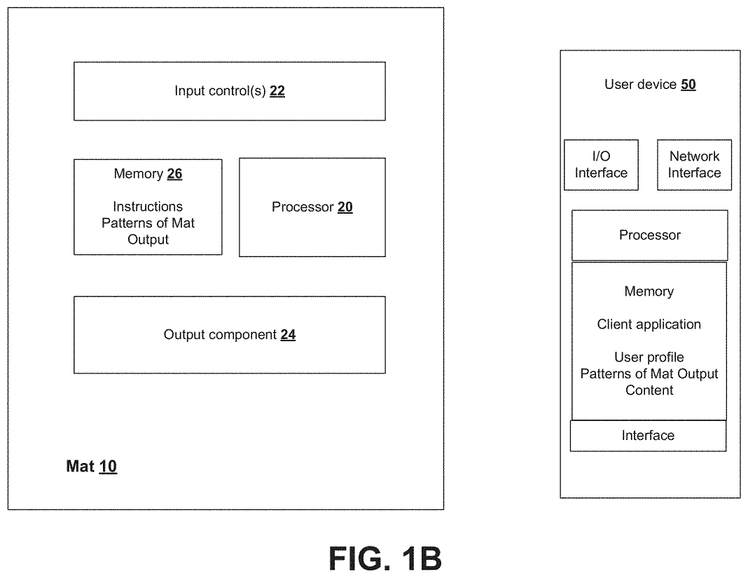

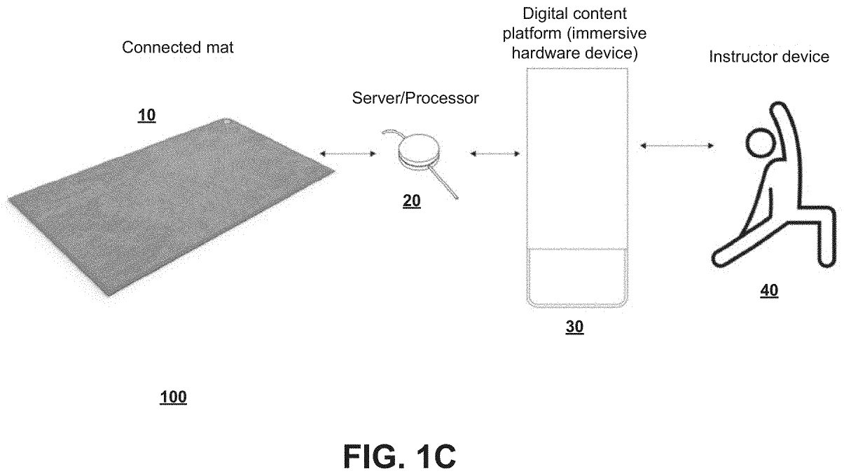

FIG. 1A shows an example embodiment of a workout mat 10 to enhance digital connection through sensory output of a physical connected workout surface of the mat responsive to input data. The workout mat 10 has at least one input control 22 to receive a selected pattern of mat output from a plurality of patterns of mat output. The workout mat 10 has at least one output component 24 to provide sensory output according to the selected pattern of mat output. The output component 24 can be integrated as part of the mat 10, or can include one or more device connected to the mat 10 that are activated or triggered by control commands. For example, the selected pattern of mat output can have pre-programmed patterns of mat output that are selectable by the user using the input controls 22 in the workout mat. For In some example embodiments, the user can use the input controls 22 in the workout mat to program and/or modify the pattern of mat output. The workout mat 10 may have a memory storing instructions for the programmed patterns of mat output.

The workout mat 10 has at least one input control 22 to receive a selected pattern of mat output from a plurality of patterns of mat output. The workout mat 10 can have different types of input controls 22. For example, an input control 22 can be a sensor integrated into the mat that can be activated by the user to select a pattern of map output. The mat 10 can have multiple sensors and sensor zones that correspond to different input controls 22. The mat 10 can be composed of different layers and, in some embodiments, one or more layers can provide input controls 22. As another example, an input control 22 can be a button integrated into the mat 10. An input control 22 can be integrated into a control unit for the mat 10 to receive instructions and control commands from different components and/or devices and to activate or trigger different patterns of map output.

A pattern of map output can indicate one or more types of sensory output, timing data for triggering or activating sensory output, location data indicating regions, zones, locations or portions of the mat 10, and so on. For example, a pattern of mat output can indicate one or more types of physical sensory output such as heating, cooling, or vibrations. Visual output and audio output are further example types of patterns of map output. A pattern of map output can also indicate different peripheral devices to actuate or activate as part of a sensory experience. The pattern of mat output can also define different times for triggering one or more types of output, and/or locations on the mat 10. The pattern of mat output can be a pre-programmed pattern of mat output, or the pattern of mat output can be programmable by the user to define the pattern of mat output. The pattern of mat output can be a modifiable pattern of mat output.

A pattern of map output can be linked to different attributes such as exercise/activity type, content, duration, sensory output type, timing, and so on. Attributes can be a combination of activity and duration. Attributes can be embedded mat outputs linked to different selected exercises and/or selected experiences. In some embodiments, a user profile can link to or define one or more attributes of pattern of map output. A client application on an electronic device has a user interface to generate the user profile to define one or more attributes of the selected pattern of mat output. As an illustrative example, a pattern of map output can indicate that a specific zone of a mat 10 be heated to a temperature for a duration of time, and then another zone of the 10 be heated to another temperature for another duration of time. The pattern of map output can also indicate that a speaker integrated into the mat 10 generate audio output for time duration. The user can be presented with different patterns of map output for selection. For example, a client application can display multiple patterns of map output at an interface and a user can select a pattern of map output using the interface. The client application can then transit control commands to the input control 22 of the mat 10 to trigger the selected pattern of map output. As another example, a user can be presented with different activities each linked to a selected pattern of map output. A user can select an activity which in turn results in the selected pattern of map output linked to the activity. In some embodiments, an activity may be linked to multiple patterns of map output. When a user selects the activity then the linked patterns of map output may be provided to the user for selection. The selected pattern of map output may be one of the patterns linked to the selected activity. The workout mat 10 can also be connected to input device(s) to receive input data to trigger content at a digital content platform. For example, the content can be pre-programmed content or user specified content. The input device can provide control commands to the input control 22 to select a pattern of map output. In some embodiments, the input control receives instructions for the sensory output from exercise content, such that the instructions are embedded within the exercise content. In some embodiments, the input control receives instructor input from an instructor device to modify the instructions for the sensory output and/or to select a pattern of map output.

As an illustrative example, the output component 24 can include one or more heating elements to generate heat as an example sensory output. The heating elements can be activated according to the selected pattern of mat output. Further examples of the output component 24, input controls 22, and patterns of map output are described herein.

In operation, a user can use the input controls 22 to select and/or modify a pattern of map output at the workout mat 10. There can be multiple patterns of map output that can be selected and/or modified by the user. The output component(s) 24 generate a sensory output based on the selected and/or modified pattern of map output. This can provided a heated sensory experience at the mat 10, for example.

The workout mat 10 enhances digital connection through sensory output of a physical connected workout surface of the mat responsive to input. The workout mat 10 can have different layers such as a non-slip textile surface layer, a multi-zone heating layer, a sensor layer, a mat base layer. One or more layers can integrate with an input control 22 and/or provide the input control 22. Further, one or more layers can integrate with an output component 24 and/or provide the output component 24. In some embodiments, the input control 22 is a low-power controller integrated into the mat 10 to consolidate signals from sensors of the sensor layer. The controller can also direct controls to zones of a multi-zone heating layer. Power and data can be transmitted to the controller by a connector. In some embodiments, the low-power controller is removable. In some embodiments, the mat 10 has a light portion that provides an indicator and a touch surface as another example input control 22. In some embodiments, the input control 22 is an input surface and a sensor layer integrating a plurality of sensors.

In some embodiments, the input control captures interactions with the connected mat 10 to trigger control commands relating to the selected pattern of pattern of mat output. The interactions can be gesture recognition, taps in zones on the mat, and intelligent activity recognition based on context.

The output component 24 provides sensory output according to the selected pattern of mat output. In some embodiments, The output component 24 synchronises the sensory output with content at a digital content platform. For example, the pattern of map output can have timing data that synchronises with timing data of the content of the digital content platform. As another example, the content can embed instructions for a pattern of map output that is synchronised with the content. In some example embodiments, the sensory output of the physical connected workout surface can be controlled or activated by exercise content of a digital content platform. The content can have one or more timestamps or metadata to synchronize the sensory output with the exercise content.

FIG. 1B shows an example system for a workout mat 10 according to embodiments described herein. In some embodiments, user device 50 (e.g. electronic device, mobile device) has a processor and a memory storing a client application executable by the processor. In this example, a user device 50 has a client application with an interface that can be used to generate a user profile for sensory output of the workout mat 10. In some embodiments, the client application can be used to select and/or modify a pattern of map output for the workout mat 10, and can provide control commands to the input controls 22 of the workout mat 10 to trigger the output component 24 to provide the sensory output at the workout mat 10. In some embodiments, the client application defines one or more user profiles to store, generate or modify different patterns of map output. For example, the client application has a user interface to receive input data to create or update user profiles to define one or more attributes of the selected pattern of mat output. For example, attributes can relate to a combination of activity and duration. As another example, the attributes can be linked different patterns of mat outputs, an exercise and/or an experience.

In some embodiments, the client application can be used to define user profiles of different activity/duration combinations (e.g. yoga activity for 30 minutes), and to select exercise content or experiences that are linked to patterns of map output. For example, the exercise content may have embedded patterns of mat outputs that can provide control commands or instructions to the input controls 22 of the workout mat 10 to trigger the output component 24 to provide the sensory output according to the embedded patterns of mat outputs linked to the exercise content or experience.

In some embodiments, the user device 50 integrates at least one hardware processor with a data storage device (including memory or other data storage elements or a combination thereof), and at least one communication interface, such as a network interface or a I/O interface. The hardware processor may be, for example, a microprocessor or microcontroller, a digital signal processing (DSP) processor, an integrated circuit, a field programmable gate array (FPGA), a reconfigurable processor, a programmable read-only memory (PROM), or any combination thereof. The memory stores executable instructions, user profiles, content, patterns of map output, trained models, and so on. The processor can execute the instructions to implement operations described herein. The memory may include a computer memory that is located either internally or externally. The I/O interface enables the processor to interconnect with one or more input devices, such as the connected workout mat 10, peripheral devices, a keyboard, mouse, camera, touch screen and a microphone, or with one or more output devices such as a display screen and a speaker. The network interface enables the processor to communicate with other components, to exchange data with other components, to access and connect to network resources, to serve applications, and perform other computing applications by connecting to a network (or multiple networks) capable of carrying data including. The client application is operable to register and authenticate users (using a login, unique identifier, and password for example) prior to providing access to user profiles. The client application may serve one connected workout mat 10 or multiple connected workout mats 10. In some embodiments, the client application can have instructions to configure the processor to process video data capturing user movements to analyze user movements to trigger selections of different patterns of mat output. In some embodiments, the client application can have instructions to provide content, such as exercise content or experience content, and send control commands to the mat 10 to synchronize the sensory output (e.g. patterns of map output) with the content.

Further details relating to the client application and interface are provided herein.

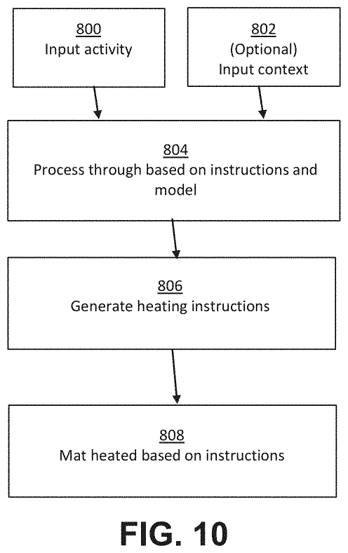

FIG. 10 shows an example system 100 for a workout mat 10 according to embodiments described herein. In some embodiments, the workout mat 10 is connected to a hardware processor 20, and may be referred to as a connected workout mat 10. The system 100 has a connected mat 10, a hardware processor 20, and a digital content platform 30. In this example, the digital content platform 30 is integrated with a immersive hardware device (such as a mirror with a display screen). In other example embodiments, the digital content platform 30 is not an immersive hardware device, and can be a cloud server or computing device, for example. The connected workout mat 10 creates sensory experiences based on selections made using input controls at the mat 10, input data or user preferences. The connected workout mat 10 has at least one input control (e.g. an input surface) to receive selections for patterns of mat output, and/or at least one input device to capture or receive input data for data exchange with the hardware processor 20 for processing. The connected workout mat 10 has at least one output component or device that generates physical sensory output to create a sensory experience according to patterns of map output.

In some examples, the connected workout mat 10 output synchronises the sensory output with content delivered through the digital content platform 30 as part of the sensory experience. The connected mat 10 has a communication channel and couples to a hardware processor 20 which in turn couples to the digital content platform 30 by a communication channel. For example, the hardware processor 20 can receive data indicating selected exercise content and provides the pattern of map output (e.g. to the at least one input control) at the mat 10 based on the selected exercise content to synchronize the selected exercise content and the sensory output. The hardware processor 20 can trigger digital output corresponding to selected exercises for the digital content platform 20 or physical sensory output at the connected mat 10 or a peripheral device connected to the system 100. The digital output and/or physical sensory output can be generated based on selections or input controls captured by an input control (e.g. input surface, buttons, interactive display) of the connected mat 10, video data captured by the digital content platform 20, and/or instructor input from an instructor device 40 or from a production studio that may process requests from an instructor. In some embodiments, sensory output can also be controlled or activated by the content alone without the user input or instructor input. For example, the content can have embedded instructions for patterns of mat output. As another example, sensory output can also be controlled or activated by the content using time stamps or metadata embedded within the content, or by using machine learning of video or instructor audio. The content can include recorded content. The mat 10 can connect with the digital content platform 30, and can also connect to external sources of content, such as different content services. For example, the mat 10 can connect with the processor 20 to access different content services, without connecting to the digital content platform 30.

Accordingly, in some embodiments, the hardware processor 20 receives selected exercise content of a digital content platform 30 and provides the pattern of map output to the mat 10 based on the selected exercise content to synchronize the selected exercise content of the digital content platform 30 and the sensory output at the connected mat 10. For example, instructions for patterns of mat outputs can be embedded in a recorded exercise program. In some embodiments, instructions for patterns of mat outputs are modified by an instructor device 40 (or production assistant) during a live exercise class. In some embodiments, the live class may be recorded as a program and later be broadcast with the instructions for patterns of mat output embedded therein. The exercise content can be in different formats, and can include audio and video content data. For example, the exercise content can be delivered in different environments such as virtual reality environments, augmented reality environments, mixed reality environments, and so on.

The digital content platform 30 can be an immersive hardware device such as an camera device and display device integrated with a mirror. However, in some embodiments, the digital content platform 30 is not an immersive hardware device. The hardware processor 20 processes the input data using a pre-trained model to generate control commands for the digital output and/or physical sensory output by defining or modifying patterns of map output. For example, the physical sensory output can involve heating, cooling, or vibrations at the connected workout mat 10 that can be invoked or triggered at different times or locations based on the pattern of map output. The digital output can involve video or music at the digital content platform 30 or display device proximate to the mat 10 that receives input data from the digital content platform 30. The digital output can involve personalized guidance and instruction at the digital content platform 30.

The hardware processor 20 can also process the input data and generate exercise metrics to provide analytics about the user's exercise or activity at the connected workout mat 10. For example, the hardware processor 20 takes input from a pressure sensor integrated with the connected workout mat 10, and translates that data into meaningful metrics and feedback that are conveyed through a display device (e.g. at digital content platform 30 or separate immersive hardware device) or through other output mechanisms integrated or coupled to the connected workout mat 10. The hardware processor 20 compares input data against pre-trained models to classify the user activity, then the processor 20 determines the appropriate intervention. In some embodiments, the processor 20 can generate a corresponding pattern of mat output or control command to create a sensory experience by digital output and/or physical sensory output at the mat 10. The trained model can process the data to distinguish control input from exercise activity (e.g. a button tap versus a squat). Moreover, the hardware processor 20 executes software or code (stored in memory) which can classify the input from the pressure sensor to determine user activity and other information. Examples include: repetition counting, repetition rate estimation, pose determination, pose stability, left and right pose balance, front and back pose balance, pressure distribution through hands, jerkiness, center of pressure, velocity of center of pressure, acceleration of center of pressure, estimations of movement quality, and so on. The hardware processor 20 can be updated to re-train and update model. Further, the hardware processor 20 complies to various protocols to integrate with other peripherals wirelessly.

The connected workout mat 10 can provide sensory output that synchronizes with content provided by a digital content platform 30 to deepen immersion and elevate a user's exercise practice, or generate a sensory experience. The digital content platform 30 can connect to or integrate with an immersive hardware device with displays integrated with mirrors and cameras to capture video data, for example. The connected workout mat 10 can trigger the capture of video data of a user during active exercise, such as a series of yoga sequences. The connected workout mat 10 can provide sensory experiences such as guided heat to engage the body and mind. The connected workout mat 10 can enable users to self-correct and connect with themselves using video playback features, as well as instructors, and other users. The connected workout mat can enable users to improve exercise by capturing input data and the processor 20 can generate exercise metrics by processing the captured input data.

The connected workout mat 10 is multifunctional to deepen immersion, provide different sensory experiences, and help users improve their practice. The system 100 captures input data from the participant, processes the input data using the processor 20 (and pre-trained model), and generates a digital output and/or physical sensory output experienced by the participant. This can involve automatic selection of a pattern of map output (from multiple patterns) to trigger the desired sensory output at the mat 10.

In some embodiments, the outputs can be controlled by a remote instructor using an instructor device 40 to generate control commands that provide direct manipulation and control over a participant's physical workout to create and control the sensory experience for a remote participant. Instructors can be recorded in a live studio with a video camera and the captured video content can be played at the digital content platform 30. The instructor can have the ability to control the temperature of a connected workout mat 10 through either interaction with their own mat, a (remote control) instructor device 40, an application, or through a signal given to a production crew.

The digital or physical sensory output experience can involve a range of outputs (e.g. heating, cooling, vibration) exhibited through the mat 10 during both live and recorded sessions. The sensory outputs at the mat 10 can be defined or triggered by instructions for a selected pattern of mat output. The pattern of mat output can also be selected and/or modified by user device 50 and/or instructor device 40. Content (of the digital content platform 30) can also be controlled through interaction with the connected workout mat 10. These interactions include gesture recognition, taps in specific or customised zones on the connected workout mat 10, and intelligent activity recognition based on the context (i.e. auto detection of meditation, HIIT, or other activities). The context can be defined by the system 100 using a number of inputs, including activity monitors, connections to peripherals, activity detection, time, location, and so on. The connected workout mat 10 serves as both an input device and output device that synchronises with content through the digital content platform 30, controlled by live or pre-recorded instructors (via instructor device 40). Instructors can orchestrate experiences that will be felt by participants during both live and pre-recorded sessions.

The custom zones of the connected workout mat 10 can be defined through an application interface that synchronises with the digital content platform 30. In this application interface, there may be an image or visual representation of the connected workout mat 10, and specific zones can be selected and customised to trigger specific outputs, control commands, or features. The pattern of map output can be linked to different zones or locations of the workout mat 10 to create different sensory outputs at the different zones at different times.

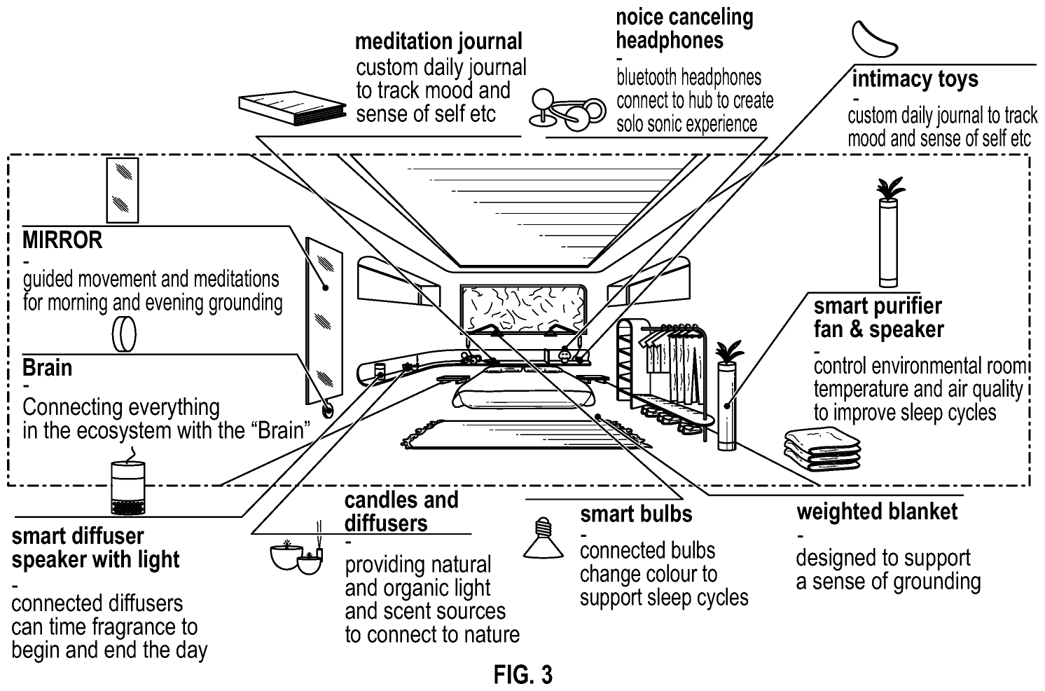

The hardware processor 20 serves as an interface between the connected workout mat 10 and the digital content platform 30, or other external devices such as user device 50 and instructor device 40. The hardware processor 20 processes the raw data from sensors (integrated with the connected workout mat 10), maintains wireless communication with the digital content platform 30 or immersive hardware device, and enables integrations with other peripherals that can be integrated into the system 100. Examples of integrations with peripherals are described further in relation to FIG. 3. The digital content platform 30 (e.g. immersive hardware device) can process the output data to control content.

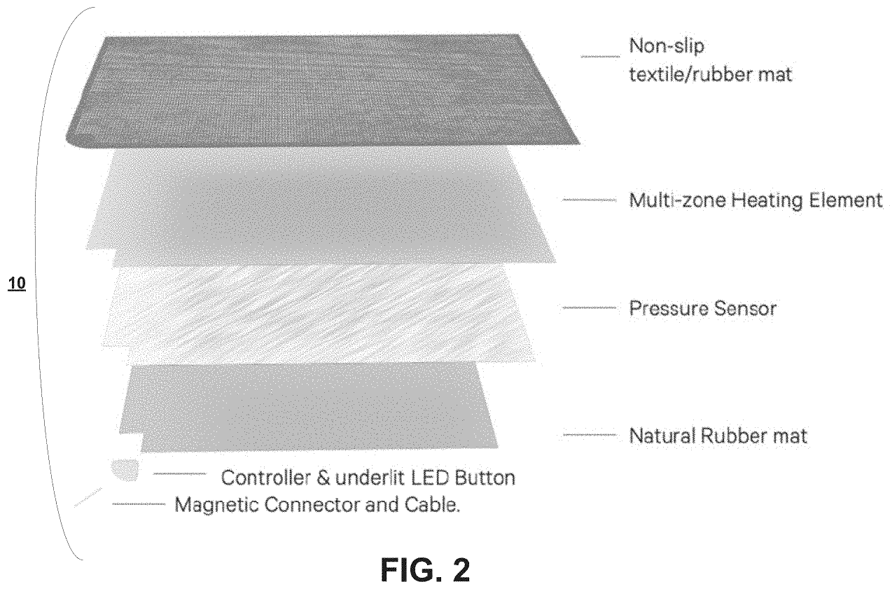

FIG. 2 shows an exploded view of the connected workout mat 10 according to embodiments described herein. The connected workout mat 10 can have different layers that can be used to implement different input controls 22 and/or output components 24. For example, the workout mat 10 can have a non-slip textile surface layer, a multi-zone heating layer, a sensor layer, a mat base layer. As shown in FIG. 2, an example architecture of the connected workout mat 10 includes a pressure sensor, one or more independent heating elements to define one or more heating zones, a non-slip top, and a natural rubber mat base. For example, the non-slip textile surface can be made of textiles with rubber deposited on the top, or three dimensional knitted composite textiles which embed rubber and textile into one layer. Example approximate dimensions of the connected workout mat 10 can be 1.8 m×1 m×7 mm thickness. These are illustrative example dimensions to provide utility in a range of different forms of exercise activities (e.g., HIIT, barre, yoga). The pressure sensor layer can provide input controls 22 (or input data) for the mat. For example, the pressure sensor layer can generate sensor input to indicate activation of different locations on the mat 10, and the different locations or activated sensor areas can indicate selection of different patterns of map output or be used to define or modify patterns of map output. The pressure sensor layer can also provide input data that can be processed to analyze user movements. The multi-zone heating element layer can provide output components 23 to generate sensory output at the mat 10. The multi-zone heating elements can be activated according to a pattern of mat output to provide different sensory outputs. The patterns of mat output can define different sensory output experiences. For example, the patterns of mat output can trigger activation of different zones of the multi-zone heating element layer at different times. The multi-zone heating elements can also be activated in synchronization with content to enhance the content experience for the user. For example, the content can embed instructions a pattern of map output (including type, location, timing) that can be used to synchronize the sensory output with the content.

The connected workout mat 10 could include a low-power, removable controller integrated into the mat 10 to consolidate signals from sensors of the sensor layer. The signals from the sensors can be input for the input control to select a pattern of map output, for example. The signals from the sensors can be input data that can be processed to analyze user movements, as another example. The controller can also activate different output components based on a pattern of map output. The controller directs control commands to zones of the multi-zone heating layer to activate heating zones based on the pattern of map output. A connector transmits power and data to the controller. In some embodiments, the connected workout mat 10 (with controller) transmits the consolidated signals to the processor 20 by the connector. In some embodiments, the workout mat 10 can have power by a battery and can transmit data wirelessly. The workout mat 100 configuration may vary to conserve battery usage, such as by have fewer heating zones or no heating zones, for example, to avoid excessive battery usage.

In the example shown in FIG. 2, the controller is integrated into the connected workout mat 10 to consolidate signals from the pressure sensor (e.g. to capture selections for input control 22, or input data) and direct control commands to various zones in the heating layer (e.g. to trigger sensory output by output components 24 according to a pattern of map output). The controller can be designed to be thin to improve the aesthetic of the mat, reduce the risk of a user tripping on the mat, and to facilitate upgrades. Power and data is transmitted to the controller by a magnetic connector and cable. The connected workout mat 10 has a light portion that provides an indicator and a touch surface. The controller can integrate with an LED button which is another example input control 22 to select a pattern of map output, for example.

The connected workout mat 10 is a multifunctional workout mat that can facilitate a range of inputs (to capture selections and input data) and outputs (to trigger sensory output) to create sensory experiences. The hardware processor 20 controls content presented at the digital content platform 30 by user interactions with an input surface of the connected workout mat 10. An example experience the input surface might enable relates to content control which can include changes to playback of the content (e.g. play, pause, skip, forward, reverse), volume of instructor, volume of music, selection of content, and selection of quick-access features, such as a trigger to record an activity for review later, and so on. The hardware processor 20 can also control selections or modifications of patterns of map output by user interactions with an input surface (e.g. input component 22) of the connected workout mat 10.

Interactions with an input surface of the connected workout mat 10 can be taps in zones of connected workout mat 10. Another example experience of the input surface of the connected workout mat 10 is zone specific control: Specific zones on the mat might be defined, designated, or customised zones that trigger specific features, patterns, controls or output.

The input surface of the connected workout mat 10 can detect a user on the mat 10, and location of a user on the mat 10. An example experience the input surface might enable is presence detection such that the mat detects when a user stands on it and the location of the user's feet on the mat. Another example experience the input surface might enable is user detection. The connected workout mat 10 detects which user is standing on the mat using trained models and/or the sensors integrated into the connected workout mat 10. The connected workout mat 10 and the processor 20 can implement user detection in different ways. For example, the connected workout mat 10 detects a user thereon. In this case, the connected workout mat 10 and the processor 20 detects pressure on the mat and determines that someone is standing on it. The detection can be a detection of a specific user. For example, the processor 20 can determine that there are two feet on the connected workout mat 10 looking towards the digital content platform 30 and then the processor 20 can classify the input data as “user detected”. The connected workout mat 10 can also detect a specific user on the mat 10. This user detection can require a specific user to step on and off the connected workout mat 10 to train the processor 20 (and train models). Every individual has a unique gait, and the nuances of this gate can be attributed to an identity and stored in a user profile in memory coupled to the processor 20. In analysing the gate, the processor 20 can generate a model that is unique to a specific individual. This can be implemented through a prompt process to request the user stand on the mat 10 to collect data, or, more covertly by passively collecting data while knowing which user is signed into a specific account while working out on the connected workout mat 10. The input surface of the connected workout mat 10 can enable an alarm mode. This experience may involve generating an alarm. Deactivating the alarm may require users to physically step on the connected workout mat 10 or in front of the digital content platform 30. For example, requiring a user to step onto the connected workout mat 10 or in front of the digital content platform 30 may increase adherence to a workout plan or schedule that the user or an instructor has set. The alarm may remind or prompt the user about the workout plan or schedule. The system can recognize that the user remained on the mat 10 (e.g. due to the pressure data captured by pressure sensors) after an activity has ended and can automatically extend your practice with add on components (e.g. meditation, restorative poses, stretches).

The input surface of the connected workout mat 10 can enable auto-activity detection. For example, the connected workout mat 10 can determine the activity based on a profile generated from input data captured by the pressure sensors, or a profile created by client application. The input surface of the connected workout mat 10 can also be used to select content for the activity or exercise. For example, a user might run on the spot to select a cardio session presented by the digital content platform 30. As another example, a user can walk towards the connected workout mat 10 and sit down on the connected workout mat 10 in the morning. Raw sensor data is transmitted to the processor 20. Once received, the processor 20 cleans and filters the data, processes this data and classifies the user's position based on pre-trained models. The processor 20 determines that the user is sitting, and understands that it is the morning by time data captured as input data. Given this input data and a preset from the user, the digital content platform 30 plays the user's favorite morning meditation session. As another example, the user the walks towards the connected workout mat 10 and starts jogging on the spot. The processor 20 receives the input data from the connected workout mat 10 and interprets the data as a signal to jump to various HIIT workouts. In this scenario, the user doing the activity (e.g. jogging on the connected workout mat 10) is the input method.

The connected workout mat 10 can trigger capture of a video recording of a user's exercise session (or portions thereof) and playback of the captured video at the digital content platform 30. The video feature can enable different functionalities, such as review of a pose (e.g. playback of video either at end of workout or during the workout by the user or the instructor to aid in corrections and understanding of form), detection of a pose (e.g. automatic recognition of movements or poses in the video) and holding a pose (e.g. when user taps button to trigger video capture and the user focuses on their pose which is captured by video). For example, touching a specific zone of the input surface on the connected workout mat 10 during a session can trigger a camera in an immersive hardware device (e.g. digital content platform 30) to record video of the user on the connected workout mat 10. The video can be reviewed by participants afterwards. An instructor can participate remotely using instructor device 40. Participants can review a video of themselves doing various poses with or without a remote instructor. When reviewing it with an instructor, the instructor and participant have the ability to scrub through a video and analyse the captured poses for improvement. The participant can also navigate through the video through interactions with the connected workout mat 10 or through an application (e.g. on a mobile device) that controls the digital content platform 30, for example. The feature can be activated through an interaction with the connected workout mat 10, or can also be activated through other input mechanisms like pose detection enabled through the connected workout mat 10, input to the application, or other markers that give insight into the pose that a current user might be in. Gesture recognition can also be used to automate aspects of the pose review by processing video data to automatically recognize gestures or poses.

Accordingly, the connected workout mat 10 and the digital content platform 30 can enable participants to review a video of themselves doing various poses with or without a remote instructor. In one embodiment, a pressure map profile can also be reviewed. The video and the pressure profile can be synchronized, for example. When reviewing it with an instructor, the instructor and participant have the ability to scrub through a video and/or the pressure profile and review the poses together for improvement. The video review can be controlled through interactions in the mat or through the mobile device that connects to the processor 20 or digital content platform 30 to send control commands to the playback video. The video playback can be activated through an interaction with the connected workout mat 10, and can also be activated through other input mechanisms such enabled through the mat, app input, or other markers that give insight into the pose that a current user might be in.

The input surface of the connected workout mat 10 can detect a user's balance and stability during an exercise session. For example, pressure sensors built into the connected workout mat 10 can enable detection of balance and centre of pressure. Pressure sensors built into the connected workout mat 10 can also enable detection of stability and jerk of the user. Jerk is a derivative of acceleration and the workout mat 10 can measure the rate of acceleration increase or decrease, or otherwise measure quality of movement. Balance can be defined in a number of ways, but in general, it can be inferred from stability. For example, a user hopping, jerk, a user putting a foot down, the sway in the pressure reading, etc. can all lead to the determination by the processor 20 that a user is unbalanced. Centre of pressure (COP) is an average that can be determined by the processor 20 from the data received from the pressure sensing elements. The COP can be determined by the processor 20 taking the raw data input and location of that input and averaging it across the area of the pressure applied. For example, one point at 0,0 can be 100 pressure, and the other point at 0,50 can be 0 pressure, then the COP can be at location 0,50, with a pressure of 50. The jerk in a user's movement can be determined by estimating postural sway and erratic movements on the connected workout mat 10. This could be bolstered with analysis of video footage by user or playback of video at instructor device 40.

The input surface of the connected workout mat 10 can enable a quality of movement assessment during an exercise session. For example, the input surface of the connected workout mat 10 can detect the quality of movement both within and between yoga poses. The quality of movement can be defined through a number of different factors including the smoothness of transitions between poses, breath rate, heart rate, perceived challenge and other biomarkers. In some embodiments, the workout mat 10 can compute a ‘movement readiness’ score whereby content is selected based on the stability and control a user's body shows in that specific session. For example, the user may be tired and fatigued and highly likely for injury so the workout mat 10 selects easier workouts, or suggests that the user take it easy.

The input surface of the connected workout mat 10 can capture user metrics as input data. For example, the input surface of the connected workout mat 10 can enable repetition count during an exercise. For example, the input surface of the connected workout mat 10 can detect the number of repetitions during a particular movement (crunches, high-knees, squats, and so on). The input surface of the connected workout mat 10 can enable a live repetition rate and provide other insights to users regarding their activity and movements during a workout through a digital content platform (e.g. exercise or fitness content). For example, if the user slows down near the end of a set, stops a set slightly short, or continues working through a break, this data can be captured and provided to the user as r insight. Captured data might be used to give feedback and encouragement to users. For example, the input surface of the connected workout mat 10 can detect the rate of movement repetitions to give participants an indications of speed of movements.

The connected workout mat 10 can determine calories burned during an exercise session. For example, the connected workout mat 10 can capture input data so that the processor 20 can estimate the caloric burn rate during a given activity, or other exercise metrics.

The input surface of the connected workout mat 10 can capture different types of selections, input data and interactions. The interactions can be gesture recognition, taps in zones of connected workout mat 10, and intelligent activity recognition based on context. The interactions can be processed as selections or other commands to control or configure aspects the sensory experience of the workout mat 10.

The input surface of the connected workout mat 10 can integrate different types of devices and technologies to provide different types of input components or devices. For example, pressure sensors enable interaction through touch. As another example, capacitance sensors enable interactions through touch and air gestures. The input surface can provide spatial location tracking through ultrawide-band technology (UWB), sonar, or laser. The input surface can provide on body location tracking by UWB, ultra high frequency (UHF), inertial measurement unit, and so on. UWB and UHF are radio based technologies, for example. The connected workout mat 10 can involve heart rate sensors, electroencephalography (EEG) sensors, blood pressure sensors. The connected workout mat 10 can capture input data to determine heart rate variability (HRV), respiratory rate, galvanic skin response, and so on. The connected workout mat 10 can capture speech and other audio input. The connected workout mat 10 can have buttons and other input devices.

The connected workout mat 10 can provide a sensory experience using different output components 24 devices. The connected workout mat 10 can provide sensory output using different output components 24 according to patterns of map output. For example, the sensory output can be linked to exercise content with embedded instructions for one or more patterns of map output. The output generated or exhibited by the connected mat 10 can control or impact the exercise experience using a range of different types of output devices (e.g. heating, cooling, vibration). The outputs components or devices are designed to stimulate a participant's senses. The output devices can include different types of devices for: heating, cooling, vibration, haptics, sound, airflow, lighting, digital interface, smell, and surface material changes. The connected workout mat 10 can have heating output with multiple heating zones along the length of the mat. For example, heating of the connected workout mat 10 can be enabled by flexible resistive ink self-regulated heating elements. For example, positive temperature coefficient heating elements can be used in the connected workout mat 10 because they self regulate, meaning they cannot overheat. They are also very thin and users cannot feel the wires which may make the connected workout mat 10 more comfortable for the user.

The connected workout mat 10 can have cooling output with specific zones in the connected workout mat 10 that cool. The cooling zones are further examples of output components 24 that provide sensory output based on patterns of map output. For example, cooling of the connected mat 10 can be enabled by a refrigerated liquid cooling system, Peltier cooling elements, air forced through perforations in the mat itself, superconductive, low heat-capacity materials embedded in the mat 10, used on the mat 10 surface, and so on. The connected workout mat 10 can have haptic output with individually controllable and selectable actuators embedded in throughout the connected workout mat 10. Examples of different types of haptic actuators include coin cell vibration motors, solenoids, electro-muscle stimulation (EMS), and so on.

The connected workout mat 10 can have sound output and auditory feedback that can be provided by one or more speakers as another example of output components 24. The speakers can be integrated in the mat 10, or connected thereto. The connected workout mat 10 and processor 20 can enables sounds to be played through the digital content platform 30, as another example. The connected workout mat 10 can have airflow output. For example, there may be micro-channels in the connected workout mat 10 to direct air through and out the connected workout mat 10 towards the user. As another example, there may be a fan type device controlled by the processor 20 and in sync with content from the digital content platform 30, similar to the connected workout mat 10. The airflow output may be provided based on a pattern of map output, or instructions embedded within content.

The connected workout mat 10 can have integrated lights, or can send control commands to lighting devices coupled thereto. The connected workout mat 10 can generate visual output on a digital interface or screen, which can be integrated as part of the connected workout mat 10 or the digital content platform 30, for example. Accordingly, lights and visual output are further examples of output components 24. The connected workout mat 10 can provide notifications, statistics, or visual form feedback at the digital interface or screen, for example. The connected workout mat 10 can generate smell output that can be controlled by the system 10 and enabled through a peripheral device. The connected workout mat 10 can generate surface material changes, such as changes in texture and friction. For example, the connected workout mat 10 can have electro-actuated textiles.

FIG. 3 shows an example system with different example peripheral devices that can couple to the hardware processor 20 and the connected workout mat 10 according to embodiments described herein. The peripheral devices can implement one or more output components 24 of mat 10 or output devices that provide sensory output according to a pattern of mat output. For example, the peripherals can generate different sensory output experiences based on control commands from the hardware processor 20, the connected workout mat 10, or the digital content platform 30. The peripheral devices can also capture input data for the system 100. The peripheral devices can implement one or more input controls 22 of mat 10 to select or modify pattern of mat output, or the peripheral devices can implement one or more input devices to capture input data for processing. Example peripherals include an electronic fitness tracker (and the mat 10 can synchronize with the fitness tracker), an electronic journal to capture textual data or image data to track mood and sentiment (that can synchronize with the fitness tracker), headphones or speakers to create sound output (that can synchronize with the fitness tracker), intimacy toys, smart purifier fan to create smell output, weighted blankets with heating elements, smart light bulbs to create light output, diffusers to create smell output, and so on. The processor 20 can couple to the peripherals to transmit control commands to control the peripherals to create a sensory experience. The digital content platform 30 can also generate content as part of the sensory experience.

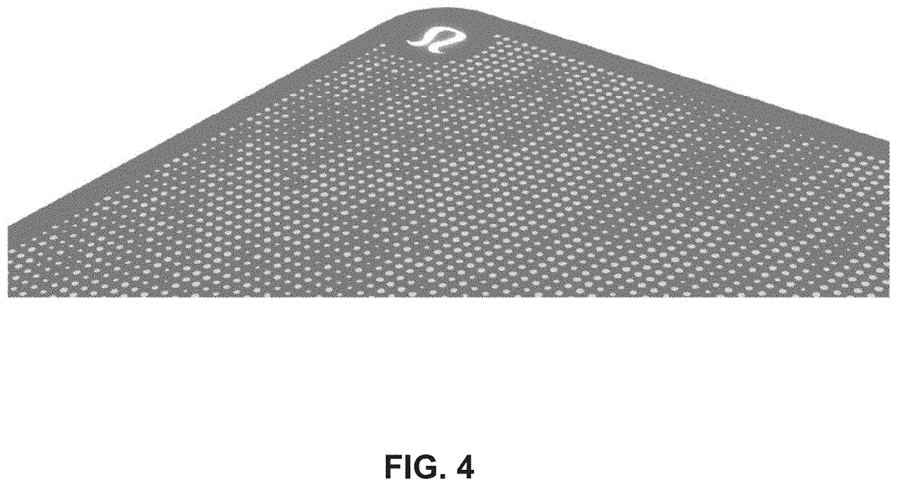

FIG. 4 shows a view of an example surface for a connected workout mat 10 according to embodiments described herein. In this example, the connected workout mat 10 has a textile surface with a dotted rubber top to provide adequate grip while maintaining the feel of a rug. The workout mat 10 has a light portion in the corner to provide sensory output. In some embodiments, the light portion can be integrated with a button as an example input component 22 to select a pattern of map output.

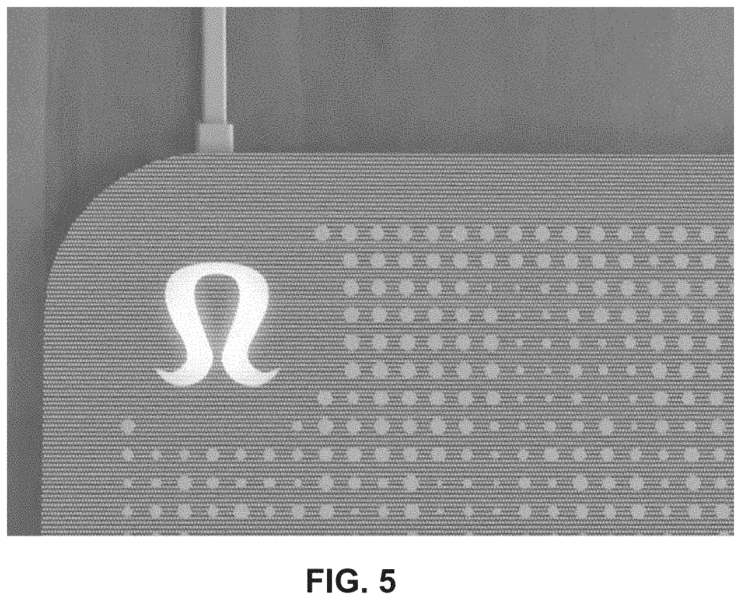

FIG. 5 shows an example connector and light portion of a connected workout mat 10 according to embodiments described herein. For example, the light portion can be an underlit LED which serves as both as a logo, an indicator to present output, and a referenceable touch surface to receive input. The LED can provide output to indicate a status, or can provide output linked to other metrics associated with a workout, like heart rate, respiratory rate, or other metrics and notifications linked to the content through the digital content platform 30 (e.g. immersive hardware device). Accordingly, the light portion can provide sensory output, and the light portion can be integrated with a button as an example input component 22 to select a pattern of map output.

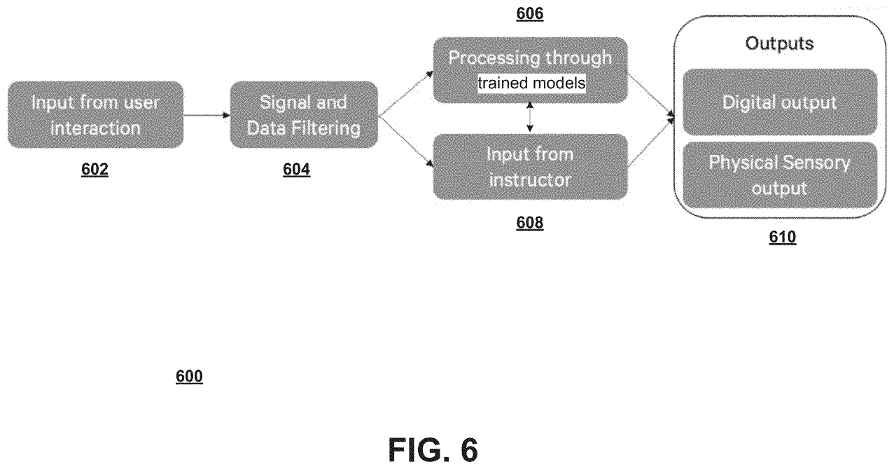

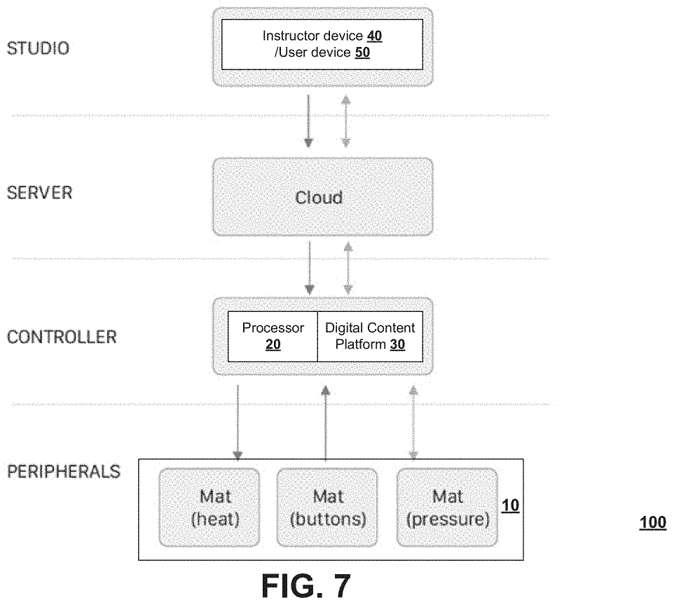

FIGS. 6 and 7 show a high level overview of the software architecture.

FIG. 6 shows an example flow diagram of a method 600 for a connected workout mat to create sensory experiences according to embodiments described herein. The method 600 can be implemented by a hardware processor 20 executing instructions stored in memory.

At 602, the processor 20 receives input data from user interactions with at least one input component 22 or input device. For example, the connected workout mat 10 can have an input surface or layer to receive input data. The input surface or layer can integrate input devices, such as pressure sensors, capacitance sensors, heart rate sensors, and so on. The connected workout mat 10 can have devices for spatial location tracking, and on body location tracking. As another example, the input data can be captured by the digital content platform 30, such as immersive hardware device with cameras to capture video data. Peripheral devices can also capture input data for the processor 20. The input can be a selection of a pattern of map output, for example. The pattern of map output can be programmed pattern of map outfit, or the pattern of map output may be defined or modified by a user based on the input data.

At 604, the processor 20 implements signal and data filtering. For example, the processor 10 can receive data from the pressure sensor (in the connect workout mat 10) to pre-process the raw data coming from the sensing elements in the connect workout mat 10. This can involve taking an array of sensor inputs and translating that into a transportable data structure like JSON or other type of data structure. The sensors (in the connect workout mat 10) create an array of (e.g. >2000) sensing elements in the connect workout mat 10 and the processor 10 converts all the values from these elements into a clean data structure. This data is then fed to the processor 10 for higher level processing. Other peripherals can connect to the processor 10 either through a wire or through wireless communications leveraging different protocols to provide input data to the processor 10 for filtering and pre-processing.

Once the processor 20 implements signal and data filtering, the method 600 can proceed to one or both of steps 606 and 608 to process the data using trained models and receive input data from an instructor. The processing can covert the data into input commands, movement data or user metrics, for example.

At 606, the processor 20 processes the input data and/or filtered signals and data using trained models. For example, the processor 20 receives input from a pressure sensor (of the connected workout mat 10) and translates that data into commands, meaningful metrics, and/or feedback that can be conveyed through a display (e.g. at digital content platform 30) or through other output components in connected workout mat 10 itself. The commands can be used to trigger selections or modifications of a patterns of map output. Inputs are compared against pre-trained models to classify the command or user activity, then the processor 20 determines the appropriate operation or intervention and can send notifications or further commands based on the operation/intervention to the connected workout mat 10 (or instructor device 40 or user device 50). The processor 20 and trained models can process the data to distinguish control input (e.g. commands) from exercise activity (e.g. a button tap on the mat 10 versus a squat recognized in the video). Moreover, the processor 20 executes software (stored in memory) which can classify the input from the pressure sensor to determine user activity and other information, such as repetition counting, repetition rate estimation, pose determination, pose stability, left or right pose balance, front or back pose balance, pressure distribution through hands or feet, jerkiness, and so on. Firmware (for the processor 10) can be updated to re-train and update the models. For example, data from activities can update the models and can train the models to be more accurate for other users. Also, the processor 10 can comply to various protocols to integrate with other peripherals wirelessly.

At 608, the processor 20 receives input data from an instructor device 40 or user device. For example, the instructor device 40 can send a command (as input data) to change a sequence of poses. The trained model can process the data to distinguish control input from exercise activity (e.g. a button tap versus a squat). The trained model can enable user detection and classify a specific user at the connect workout mat 10.

At 610, the processor 20 generates output for at least one output component of the mat 10 or device connected thereto. The processor 20 generates output data based on the results of the trained models. The processor 20 can also generate output data based on the input data from the instructor device 40. The processor 20 can synchronize the sensory output with content presented by the digital content platform 30. The hardware processor 20 generates digital output for the digital content platform 30 or physical sensory output at the connected mat 10 based on the processed input data from the connected mat 10 and/or instructor input. The output components or devices of the connected mat 10 can include different types of devices for: heating, cooling, vibration, haptics, sound, airflow, lighting, digital interface, smell, surface material changes, ambient temperature and conditions in the home, and so on. The output components or devices of the connected mat 10 can be activated based on a pattern of map output. The output can enable different experiences based on control commands or the input data (captured at the input surface of the connect workout mat 10), such as pattern selection, pattern modification, content control, zone specific control, presence detection, user detection, alarm mode, activity detection, pose detection, quality of movement assessments, and so on.

FIG. 7 shows another example architecture diagram of a system 100 for a connected workout mat 10 according to embodiments described herein. In this example, the connected workout mat 10 integrates with different peripheral devices as example input controls 22 and output components 24, such as heating devices, lights, buttons, and pressure sensors. The connected workout mat 10 transmits input data to the processor 20 and the digital content platform 30. The processor 20 can generate output by processing the input data using the trained models. The processor 20 can also process the input data using data and signal filtering processes. The processor 20 and the digital content platform 30 can exchange data with a cloud server. For example, the cloud server can generate control commands that can be transmitted to the processor 20 and in turn to the connected workout mat 10. As another example, the cloud server can transmit content to the digital content platform 30. The instructor device 40 can transmits control commands to the cloud which can in turn be transmitted to the processor 20 and the connected workout mat 10 to impact the sensory experience. As a further example, the cloud server can transmits models to the processor 20 which can be used to process input data from the connected workout mat 10.

The embodiments of the devices, systems and methods described herein may be implemented in a combination of both hardware and software. These embodiments may be implemented on programmable computers, each computer including at least one hardware processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Embodiments described herein can be implemented by servers, services, interfaces, portals, platforms, or other systems formed from computing devices. It should be appreciated that the use of such terms is deemed to represent one or more computing devices having at least one hardware processor configured to execute software instructions stored on a computer readable tangible, non-transitory medium. For example, a server can include one or more computers operating as a web server, database server, or other type of computer server in a manner to fulfill described roles, responsibilities, or functions.

The embodiments described herein are implemented by physical computer hardware, including computing devices, servers, receivers, transmitters, processors, memory, displays, and networks. The embodiments described herein provide useful physical machines and particularly configured computer hardware arrangements. The embodiments described herein are directed to electronic machines and methods implemented by electronic machines adapted for processing and transforming electromagnetic signals which represent various types of information. The embodiments described herein pervasively and integrally relate to machines, and their uses; and the embodiments described herein have no meaning or practical applicability outside their use with computer hardware, machines, and various hardware components. Substituting the physical hardware particularly configured to implement various acts for non-physical hardware, using mental steps for example, may substantially affect the way the embodiments work. Such computer hardware limitations are clearly essential elements of the embodiments described herein, and they cannot be omitted or substituted for mental means without having a material effect on the operation and structure of the embodiments described herein. The computer hardware is essential to implement the various embodiments described herein and is not merely used to perform steps expeditiously and in an efficient manner.

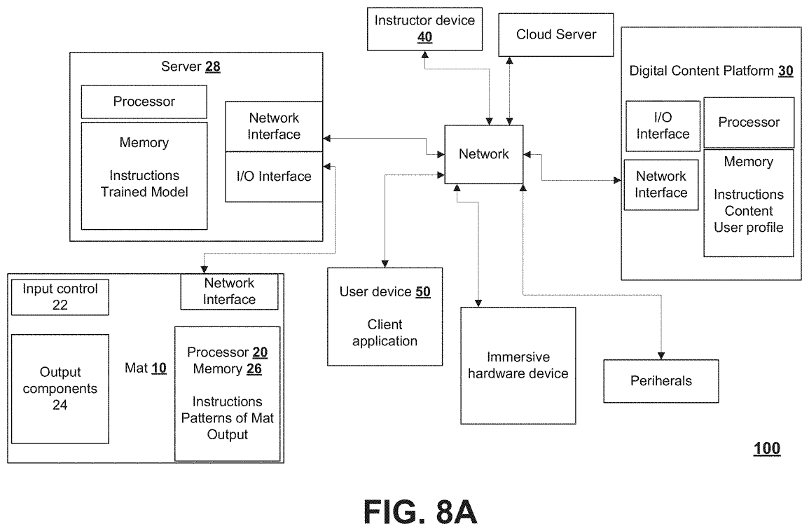

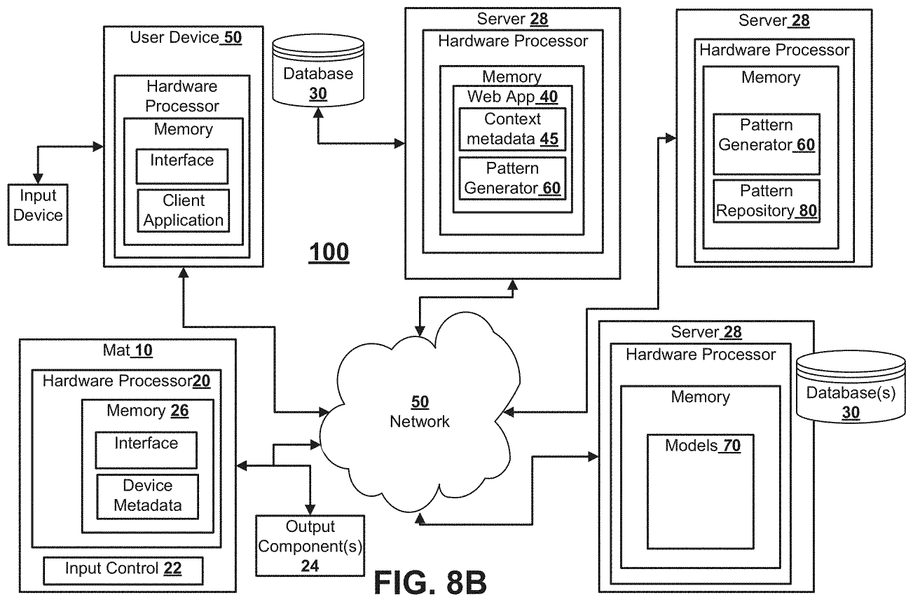

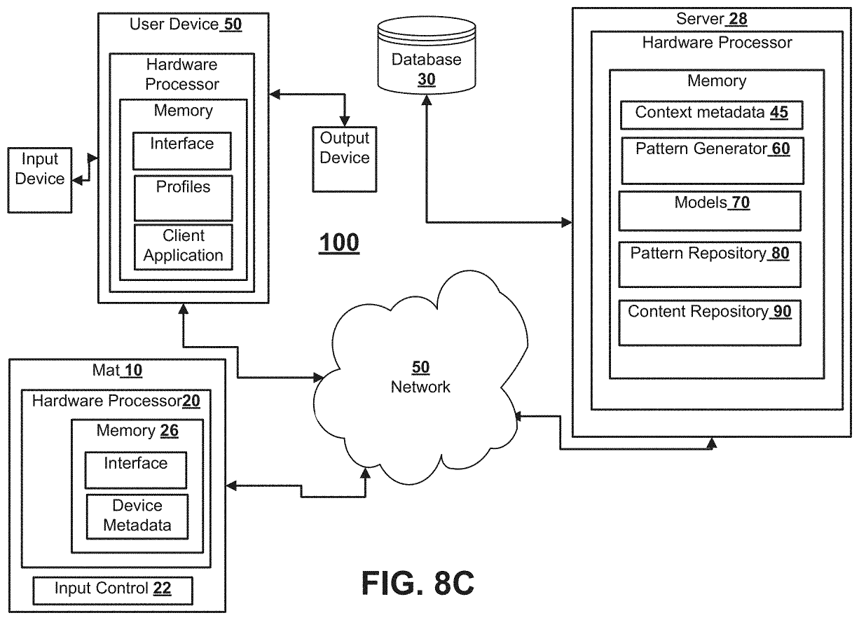

FIG. 8A shows another example architecture diagram of the system 100 with a server 28, connected workout mat 10, user device 50 with a client application having an interface, cloud server, instructor devices, peripherals, immersive hardware device (with a display screen and camera) and digital content platform 30 according to embodiments described herein. The system 100 components may be connected in various ways including directly coupled, indirectly coupled via a network, and distributed over a wide geographic area and connected via a network. In some embodiments, the processor 20 can be integrated in the mat 10, and may not be a separate unit. In some embodiments, the processor 20 can be separate and enable users to control or use peripherals independently, without a need to connect with the mat 10. Further, in some embodiments, the processor 20 can be embedded in the mat 10, and may be removable from the mat 10.

In some embodiments, user device 50 has a processor and a memory storing a client application executable by the processor. The client application has a user interface that generates a user profile to define one or more attributes of the selected pattern of mat output. For example, attributes can relate to a combination of activity and duration. As another example, the attributes are embedded mat outputs linked to a selected exercise and/or a selected experience. Further details of the client application are provided herein.

The system 100 has a computing device that integrates at least one hardware processor 20 with a data storage device (including volatile memory or non-volatile memory or other data storage elements or a combination thereof), and at least one communication interface, such as a network interface or a I/O interface. For example, and without limitation, the computing device may be a server, network appliance, set-top box, embedded device, computer expansion module, mobile device, or any other computing device capable of being configured to carry out the methods described herein.

The hardware processor 20 may be, for example, a microprocessor or microcontroller, a digital signal processing (DSP) processor, an integrated circuit, a field programmable gate array (FPGA), a reconfigurable processor, a programmable read-only memory (PROM), or any combination thereof. The memory stores executable instructions and trained models. The processor 20 can execute the instructions to implement operations described herein. The memory may include a computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. In some embodiments, the cloud server can store the trained models to reduce computational power needed at the mat 10 or in the processor 20. In this way, more complex computations can be executed in the cloud server and the result (e.g. trained models) can be returned efficiently to the mat 10 or processor 20. The memory can store instructions for different patterns of map output, for example.

The I/O interface enables the processor 20 to interconnect with one or more input devices, such as the connected workout mat 10, peripheral devices, a keyboard, mouse, camera, touch screen and a microphone, or with one or more output devices such as a display screen and a speaker. The network interface enables the processor 20 to communicate with other components, to exchange data with other components, to access and connect to network resources, to serve applications, and perform other computing applications by connecting to a network (or multiple networks) capable of carrying data including.

The processor 20 is operable to register and authenticate users (using a login, unique identifier, and password for example) prior to providing access to applications, a local network, network resources, other networks and network security devices. The processor 20 may serve one connected workout mat 10 or multiple connected workout mats 10.

As shown, the digital content platform 30 can also have at least one hardware processor, a data storage device (including memory storing instructions, content, user profiles), and at least one communication interface, such as a network interface or a I/O interface. For example, and without limitation, the digital content platform 30 may be an immersive hardware device, a server, network appliance, set-top box, embedded device, computer expansion module, mobile device, or any other computing device capable of being configured to carry out the operations described herein.

Immersive hardware device can have a display device to deliver content (e.g. exercise content) and can have other output components (e.g. speakers) to provide sensory output to user. Immersive hardware device can have input devices such as a camera to capture input data. There can be one or more peripherals to provide input devices to capture input data and/or output devices to provide sensory output.

FIG. 8B shows another example architecture diagram of the system 100 with multiple servers 20, connected workout mat 10, user device 50 with a client application having an interface according to embodiments described herein. In this example, servers 20 implement different aspects of embodiments described herein. Servers 20 can be connected by one or more networks to provide a distributed computing system. For example, a server 28 can have a web application 40 with context metadata 45 and pattern generator 60. Another server 28 can have a pattern generator 60 and a pattern repository 80. Another server 28 can store models 70 and have a database 30 of content.

FIG. 8C shows another example architecture diagram of the system 100 with a server 28, connected workout mat 10, user device 50 with a client application having an interface according to embodiments described herein. In this example, servers 28 implement different aspects of embodiments described herein. A server 28 can have a hardware processor and memory storing context metadata 45, pattern generator 60, models 70, a pattern repository 80, and a content repository. The user device 50 can have one or more input devices and one or more output devices. The user device 50 can connect with mat 10 to provide control commands (received via input device) to input control 22 of the mat 10 (e.g. selection or modification of a pattern of map output), and to receive control commands for output device to provide sensory output for mat 10.

Accordingly, embodiments described herein provide one or more systems 100 for a workout mat to create sensory experiences. The system 100 has a workout mat 10 with at least one input control 22 to receive input data for a pattern of map output and at least one output component 24 to provide a sensory output according to the pattern of map output. The system 100 can have a server 28 with a hardware processor that receives and/or stores context metadata 45 (e.g. selected exercise content of a digital content platform). In some embodiments, the server 28 with the hardware processor can identify and retrieve one or more patterns of map output using a pattern repository 80. In some embodiments, the server 28 with the hardware processor can also generate one or more patterns of map output using pattern generator 60. The patterns may be generated based on content data, user input, instructor input, and other data sets. The patterns can be stored in the pattern repository 80. The server 28 provides the pattern of map output to the at least one input control 22. For example, the server 28 can provide the pattern of map output to the at least one input control 22 based on the selected exercise content to synchronize the selected exercise content of the digital content platform and the sensory output at the connected mat.

In some embodiments, the system 100 has a user device 50 with client application with a user interface to generate a user profile to define one or more attributes for the pattern of mat output. The system 100 can generate one or more patterns of mat output using the attributes, data in the user profile, and pattern generator 60. The system 100 can retrieve one or more patterns of mat output using the attributes, data in the user profile, and pattern repository 80. The system 100 can also store patterns of map output on memory 26 of mat 10.

In some embodiments, the hardware processor processes the input data using a trained model. In some embodiments, the hardware processor updates the trained model based on data captured from other users. In some embodiments, the hardware processor trains the trained model based on user data.

In some embodiments, the system 100 can involve a digital content platform and/or an immersive hardware device, such as a mirror with a camera, display, processor and other sensors.

In some embodiments, the system 100 controls content by interactions with the connected mat 10 captured by at least one input control 22 to trigger control commands relating to the pattern of mat output. The interactions can be gesture recognition, taps in zones on the mat, and intelligent activity recognition based on context.

In some embodiments, the system 100 has an instructor device (as another example of user device 50) that provides control commands to the server 28 or mat 10 to modify the pattern of map output. In some embodiments, the output device 24 is controlled by a remote instructor device (e.g. user device 50) for direct manipulation and control a workout experience through a range of outputs comprising heating, cooling, and vibration exhibited through the connected mat.

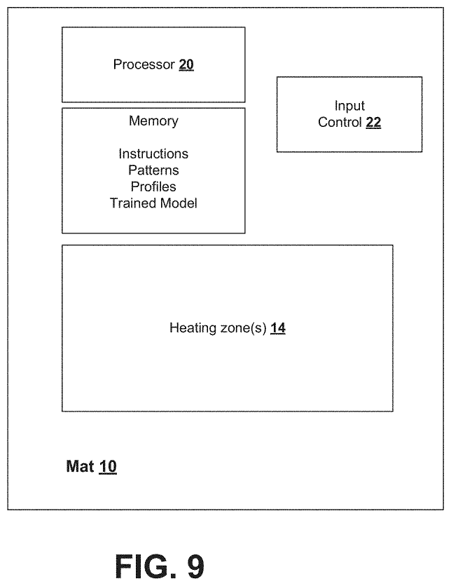

FIG. 9 is another example of an architecture diagram for a workout mat 10 with at least one heating regions or zones 14 as example output components according to some embodiments. In this embodiment, the workout mat 10 has multiple heating zones, and automated control to activate the zones according to a pattern of map output or to synchronize with content, such as an exercise program. Although the workout mat 10 may be part of a larger system, configuration, or network, with connections to other components, in some embodiments the workout mat 10 is a standalone device that does not communicate with external system components. For example, the workout mat 10 can embed an internal processor 20 (memory, instructions, models). The processor 20 and memory may be removable components, for example. The workout mat 10 can also have transceivers and a network interface to communicate with external components (e.g. cloud server, peripherals) in some embodiments. The workout mat 10 has at least one heating zone 14 or heating layer (of one or more heating elements) to provide a sensory output for the user. The workout mat 10 also has one or more input controls 22 to receive commands or input data. The input control 22 can be used to select a pattern of map output which can trigger the at least one heating zone 14 to be activated according to the pattern of map output, for example. For example, the pattern of mat output can indicate a temperature and heating duration for the zones 14, and which specific zones 14 should be activated and for how long. The pattern can be modified via control commands. The heated workout mat 10 can have a single heat region 14 or multiple-zone heat regions 14 in various embodiment and may or may not be connected.

The workout mat 10 with one or more heating region 14, can optionally have one or more pressure sensors which can capture input data or implement the input control 22. The workout mat 10 has a processor 20, memory storing instructions, patterns of map output, and trained model, optional user profiles, input/output device(s), and an optional network interface. The workout mat 10 uses its internal processor 20 to evaluate user activity (e.g. captured by input/output devices) and determine a selected pattern of map output which can indicate whether to heat the mat 10 and which areas of the workout mat 10 need to be heated to best support the user's physical activity. This support could include specific knowledge around heating and cooling locations of specific muscles and soft tissues to support the user based on the exercise or series of exercises. The exercise or series of exercises could be determined based on stored exercises in the memory, accessed by the processor 20, input or processing of an external exercise routine, or input from other connected devices or instructor system. In some example embodiments, the workout mat 10 can have temperature sensor(s) to monitor the temperature of the mat 10 and heating region 14. There can be a temperature threshold that can trigger a safety shut off or reduction of heat from the heating region 14 based on the detected temperature.

Accordingly, the workout mat 10 can enhance digital connection through sensory output of a physical connected workout surface of the mat 10 and heating zones 14 responsive to input. The workout mat 10 has at least one input control 22 to receive a selected pattern of mat output from a plurality of patterns of mat output. The mat 10 has at least one heating zone 14 (as an example output component 24) to provide sensory output according to the selected pattern of mat output. The patterns can triggers different heating zones 14 at different times and for different temperatures, for example.

In some embodiments, the plurality of patterns of mat output comprise at least one selectable pre-programmed pattern of mat output. In some embodiments, the plurality of patterns of mat output comprise at least one modifiable pattern of mat output. For example, a user can modify the temperature, location, and/or timing for the heating zones 14.

In some embodiments, a client application on an electronic device has a user interface to generate a user profile to define one or more attributes of the selected pattern of mat output. In some embodiments, the one or more attributes comprise a combination of activity and duration. In some embodiments, the one or more attributes are embedded mat outputs linked to at least one of a selected exercise and a selected experience. For example, the activity can be yoga and the heating zones 14 can generate heat for the yoga session.

In some embodiments, the mat is connectable to at least one input device to receive input data to trigger content at a digital content platform. The content can be programmed content and/or user specified content. For example, the content can be a yoga program to guide the user through the yoga session. The content can embed patterns with instructions for the heating zones 14. In some embodiments, the input control 22 receives instructions for the sensory output from exercise content, the instructions being embedded within the exercise content.

In some embodiments, the input control 22 receives instructor input from an instructor device to modify the instructions for the sensory output. For example, an instructor device can trigger or actuate the heating zones 14. In some embodiments, the at least one output component synchronises the sensory output with content at a digital content platform. For example, the sensory output of the heating zones 14 can be synchronized with the content.

In some embodiments, the sensory output of the heating zones 14 of the physical connected workout mat 10 is controlled or activated by exercise content of a digital content platform. The content has one or more timestamps or metadata to synchronize the sensory output with the exercise content.

In some embodiments, the mat 10 has a plurality of layers such as a non-slip textile surface layer, a multi-zone heating layer, a sensor layer, a mat layer. In some embodiments, the mat has a light portion that provides an indicator and a touch surface. In some embodiments, the input control 22 comprises an input surface and a sensor layer integrating a plurality of sensors.

In some embodiments, the input control 22 comprises a low-power controller integrated into the mat to consolidate signals from sensors of the sensor layer, and direct controls to the heating zones 14. Power and data is transmitted to the controller by a connector. In some embodiments, the low-power controller is removable.

In some embodiments, the at least one input control 22 captures interactions with the connected mat to trigger control commands relating to the selected pattern of pattern of mat output and the heating zones 14. The interactions can be gesture recognition, taps in zones on the mat, and intelligent activity recognition based on context.

In another aspect, embodiments described herein provide a mat 10 with heating zones 14 for providing a user with a sensory heat response to support an activity. The system has a workout mat 10 with a heat region or heating zones 14. The input control 22 can receive input data for a pattern of map output relating to the heating zones 14 to control the heat in the workout mat 10. The mat 10 has non-transitory memory storing activity data for recommended heating for an activity. The mat 10 has (or connects to) a hardware processor programmed with executable instructions to receive information about the activity, determine heat models based on the activity and generate heat instructions for the heating zones 14 of the workout mat. An output component controls the heating zones 14 in the workout mat 10 based on the heat instructions and the pattern of map output. In some embodiments, the workout mat 10 comprises non-slip textile surface layer, a multi-zone heating layer, a sensor layer, a mat layer. In some embodiments, the hardware processor determines an ambient temperature of the location of the workout mat as an input to generating the heat instructions.

In some embodiments, an activity comprises a series of related activities.

In some embodiments, an activity is associated with an activity type.

In some embodiments, the heat instructions comprise at least one of heating a heat region to more than one temperature during the duration of the activity, cooling a heat region to more than one temperature during the duration of the activity. In some embodiments, the executable instructions determine the activity from one or more activities of a connected digital platform input. In some embodiments, the executable instructions determine the activity from a pre-recorded activity input. In some embodiments, the executable instructions determine the activity from an audio input. In some embodiments, the executable instructions determine the activity from stored information that comprises at least one of a standard series for an activity type, a repetitive series, a progressive series.

In some embodiments, the non-transitory memory and hardware processor 20 are embedded within the exercise mat 10. In some embodiments, the at least one input control 22 comprises an input surface and a sensor layer integrating a plurality of sensors.

In some embodiments, the mat has a low-power controller integrated into the mat to consolidate signals from sensors of the sensor layer, and direct controls to the heating zones 14, wherein power and data is transmitted to the controller by a connector.

In some embodiments, the at least one input control 22 captures interactions with the workout mat 10 to trigger the heating zones 14. The interactions can be gesture recognition, taps in zones on the mat, and intelligent activity recognition based on context.

FIG. 10 is an example of a process flow diagram for a workout mat 10 with at least one heating zone or region. The mat 10 heating can be based on activity processing logic which can be linked to a pattern of mat output. The detected activity can be used to select a pattern of mat output from a repository of patterns, for example. The activity can also be linked to content with embedded instructions for a pattern of mat output, as another example. The activity can be a series of activities detected at the mat 10. For example, an activity can be defined as a duration of activity and of a series of activities, including patterns within the series of activities. For example, the activity processing logic can select a pattern of mat output which can trigger warming the mat 10 and cooling the mat 10 based on an activity duration. The heating of the mat 10 can support muscle activity for a specific exercise-activity. At 800, input activity is captured by the mat 10. At 802, input context is captured by the mat 10. The input context can include user preference, ambient temperature, time of day, instructor input, community input, and so on. For example, a context input might increase the temperature to support an instructor indicating a hot yoga class. A portion of the input data can be provided by a connected architecture in some embodiments. At 804, the mat 10 can process the input data (input activity and input context) based on instructions and models stored at the memory. At 806, the mat 10 (with the embedded processor 20) can generate or select a pattern of map output which can include heating instructions for the heating elements based on the processed input data. The patterns of map output can be linked to different activities and durations. At 808, the heating elements can heat the mat 10 based on the heating instructions of the pattern of mat output.

Accordingly, embodiments described herein provide one or more methods for a connected workout mat 10 to create sensory experiences.

In some embodiments, the method can provide a user with a sensory heat response at 808 to support an activity. The method involves receiving input data from at workout mat by at least one input control, and receiving input data identifying an activity. This can be input from 800 and/or 802. The method involves generating heat instructions for a heat region of the workout mat based on the activity at 806, and heating the heat region of the workout mat based on heat instructions at 808. In some embodiments, the heat region comprises multiple heat-regions receiving heat instructions. In some embodiments, the heat instructions involve code for heating the heat region to more than one temperature during the duration of the activity.

In some embodiments, the method involves generating the heat instructions by processing the input data by a hardware processor using a trained model at 804.

In some embodiments, the method involves identifying an activity from stored information that comprises at least one of a standard series for an activity type, a repetitive series, or a progressive series. In some embodiments, an activity comprises a series of related activities. In some embodiments, the method involves associating a time duration with the activity. Accordingly, the method can generate heat instructions based on the activity. In some embodiments, the method involves generating heat instructions based on the time duration associated with the activity. In some embodiments, the activity is associated with an activity type. In some embodiments, the input data identifying the activity is received from a connected digital platform. In some embodiments, the input data identifying the activity is received from a pre-recorded activity. In some embodiments, the input data identifying the activity is received from an audio input.

In some embodiments, at least one input control captures interactions with the workout mat as input. The interactions can be gesture recognition, taps in zones on the mat, and intelligent activity recognition based on context.

In some embodiments, method can involve receiving a selected pattern of mat output from a connected mat 10 having at least one input control. The method can involve generating sensory output by at least one output component of the connected mat based on the selected pattern of map output.

In some embodiments, the method involves providing a plurality of selectable programmed patterns of mat output to receive the selected pattern of mat output as part of instructions for an input activity (800) or as input context (802).

In some embodiments, the method involves receiving at least one modification for the selected pattern of mat output from a client application, modifying the selected pattern of mat output based on the modification, and generating the sensory output at the connected mat based on the modified selected pattern of map output. The sensory output can be heating the mat 10 (808) for example.

In some embodiments, the method involves determining the selected pattern of map output by signal and data filtering of input data by a hardware processor; and processing the input data by the hardware processor using a trained model (804). In some embodiments, the method involves updating the trained model over time based at least on one dataset where the dataset relates to at least one of user specific data, household specific data, ambient environment data, community data, expert data, instructor data, user goal data, community goal data, instructor goal data. In some embodiments, the method involves training the trained model with data from at least one of community data, expert data, instructor data.

In some embodiments, the method involves controlling the sensory output by interactions with the connected mat captured by the at least one input control. The interactions can be gesture recognition, taps in zones on the mat, and intelligent activity recognition based on context. In some embodiments, the method involves receiving control commands as input (800, 802) from an instructor device for the selected pattern of map output.

In some embodiments, the method involves providing exercise content from at least one of the digital content platform and synchronizing the sensory output with the exercise content provided by the digital content platform.

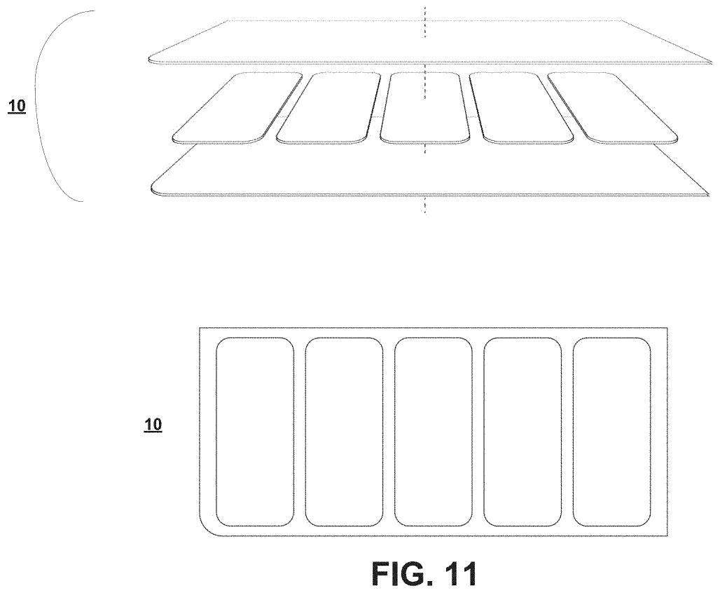

FIG. 11 is a plan view and an exploded view of a workout mat 10 diagram depicting the workout mat 10 with a multi-zone heating layer or element. The workout mat 10 has a heating layer with a plurality of zones or regions that generate heat. The heating zones or regions can independently generate heat in response to control commands or instructions of patterns of mat output. The workout mat 10 can also have a textile layer (e.g. non-slip textile, rubber). In an embodiment, the heating zones or regions may be overlapping, this is added to provide more context for heating mat 12 but applies to the layers of both 10/12 in certain embodiments.

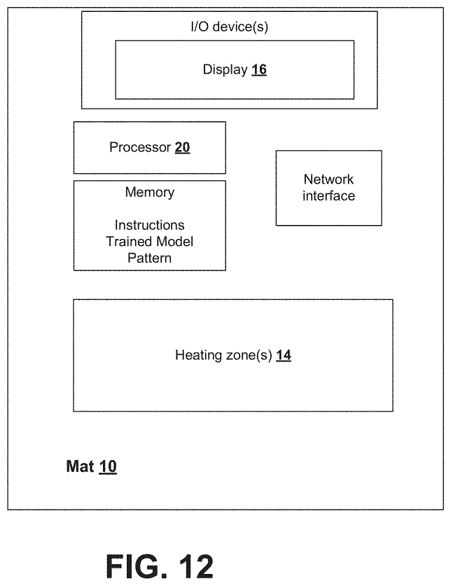

FIG. 12 shows an example architecture diagram for a workout mat 10 according to embodiments described herein. The workout mat 10 has a touch display 16 (as an example input/output device). The mat 10 optionally can have a network interface (e.g. the connector) to provide a connected mat according to some embodiments. The workout mat 10 can also be a standalone mat without the network interface in some embodiments. The display 16 can have a screen embedded in the mat 10 surface. The display 16 can be an electronic ink display or an electronic ink display with ability to be receive control commands through touch, for example. The display 16 can be in a top corner of the mat 10, for example. The display 16 can also be in different locations on the mat in other embodiments. The display 16 can have an interface with visual elements to show the temperature of the mat 10, connectivity status, other metrics, and to provide sensory output (e.g. image data). The display 16 can also include a touch screen surface which could either replace or augment the on-mat input controls 22 (e.g. other types of input/output devices).

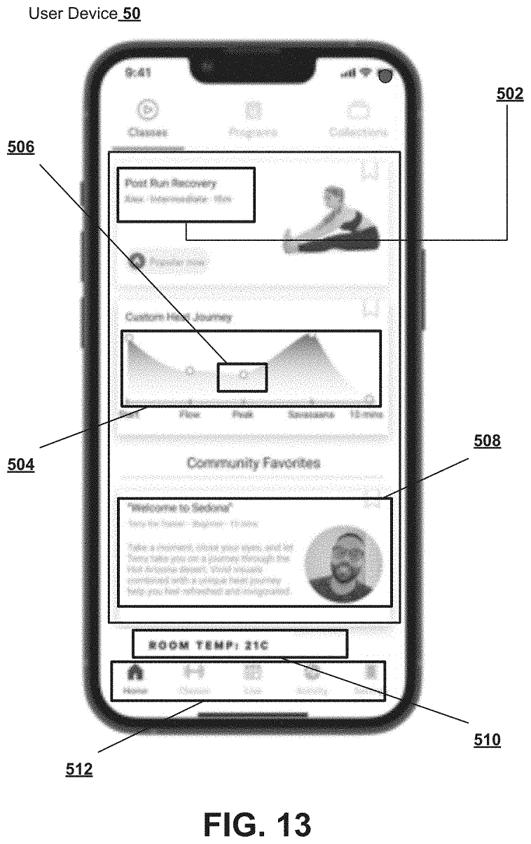

FIG. 13 shows an example user device 50 according to embodiments described herein. The user device 50 executes a client application to generate an interface with different visual elements and controls. The interface has an exercise selection portion 502 to select an exercise program or content having embedded instructions for a pattern of map output.

The interface has a heat pattern portion 504 to define heat/output journey with duration. In an example embodiment, the heat/output journey is a user defined journey (e.g. pattern of heat output) not associated to an exercise program or content with embedded instructions. The heat pattern portion 504 can be used to define patterns of heat output has one or more heat/output. The pattern can be used by the mat 10 to control heating zones and elements to generate sensory output. The heat pattern portion 504 has one or more selectable input elements 506 to modify aspects of the patterns of heat output for the mat 10. A user can drag input elements 506 (e.g. heat points) in a heat journey of the heat pattern portion 504 to change contours, for example.

The interface can have an experience portion 508 to define aspects of patterns of map output for different experiences. For example, the experience portion 508 to define other non exercise experiences. The experience may or may not have additional media (music, video, virtual reality elements, etc.) associated with the patterns of map output to trigger different sensory output experiences.

In some embodiments, the interface can have a temperature portion 510 to define aspects of patterns of map output based on one or more room temperatures. The temperature portion 510 may receive one or more room temperatures as an input for defining a pattern of map output such that the received temperature input effects sensory outputs.

In some embodiments, the interface can have other selectable elements 512 that trigger additional application functions, settings, history, etc. For example, a selectable element 512 can trigger a display of different exercise classes or programs, as an example of content that can be enhanced with the sensory output.

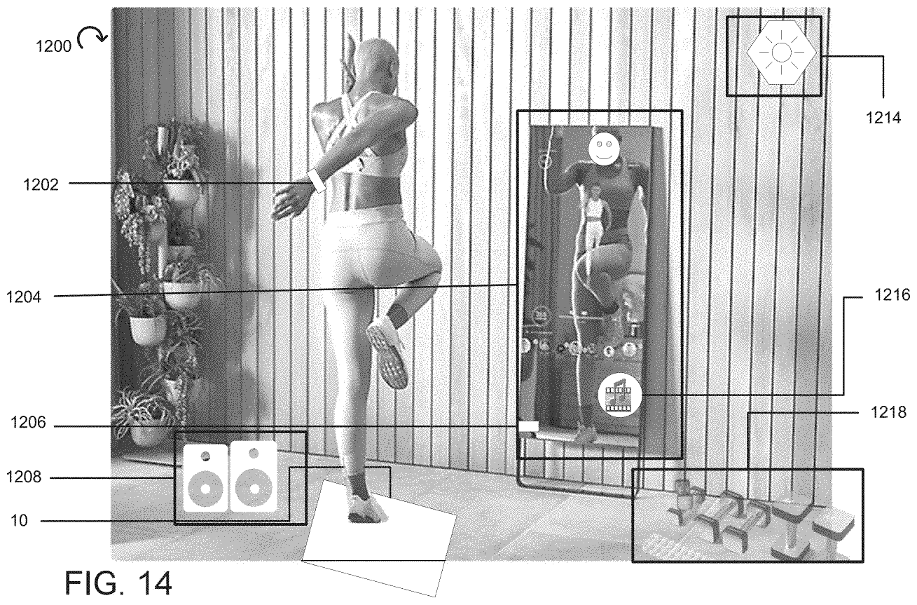

FIG. 14 shows another example illustration of system 1200 that can provide a sensory experience for exercise or activity content according to embodiments described herein. The example illustration shows different peripherals that can function as input devices to collect input data and/or function as output devices that can provide sensory output. For example, a user may be wearing a smart wrist device 1202 such as heart rate monitor that can collect input data about the user. Example peripherals are smart weights 1218 that can collect input data about movements and weight related metrics (e.g. repetitions, duration, speed). Further example peripherals that can provide sensory output (triggered based on patterns) include speakers 1208 and lighting devices 1214. There can also be an immersive hardware device 1204 that has input control 1206 (to e.g. select a pattern of map output), a display device to provide video content (e.g. exercise program) and audio 1216, and that can also have sensors, such as a camera, to collect input data. The mat 10 has a rollable surface.

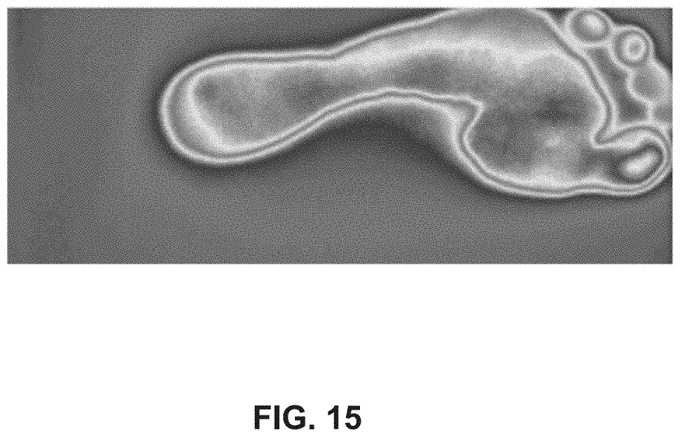

FIG. 15 shows an example portion of a mat 10 that is configured to provide visual output, such as a visual representation of heat from one or more heating elements of the mat 10. In this example, the mat 10 provides an analog visual representation of heat. A surface treatment can be applied to the top surface of a mat 10 to visually inform the user on the current relative temperature of the mat 10. The treatment can be applied in a specific pattern to represent different heat settings available on the heated mat 10 (e.g. low, medium, high).

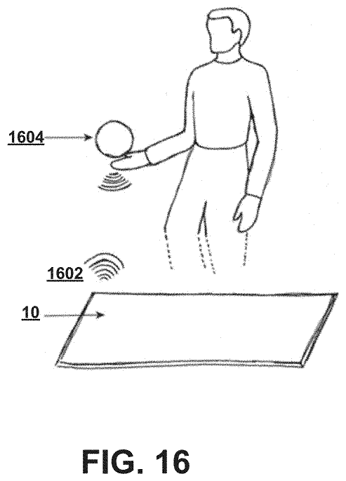

FIG. 16 shows an example mat 10 with a mat antenna 1602 that can couple wirelessly to one or more peripherals, such as an instrumented object 1604, to collect input data for further processing to generate different metrics or trigger different outputs. For example, the mat antenna 1602 can provide the ability to measure metrics (e.g. repetitions) relating to an instrumented object 1604 and also collect input data that can be used to derive information such as velocity and acceleration of specific movements through the use of radiofrequency technology, such as Ultra High Frequency (UHF) technology. The external mat antenna 1602 can be designed robustly enough such that it can be incorporated into a bendable and rollable surface of a mat 10 (e.g. a mat 10 with dimensions 200 cm×60 cm×1 cm) while still allowing the ability to measure participants above the surface (e.g. up to 3.6 meters). The user can be able to roll the full mat 10. The mat antenna 1602 can collect input data that can be used to record and detect when a repetition has occurred when a user interacts with an instrumented object 1604. The mat antenna 1602 can output a signal in real-time. The mat antenna 1602 can track multiple tags on different objects 1604, such as radiofrequency (RFID) tags. The input data collected by the mat antenna 1602 can be used by mat 10 to extrapolate velocity from the input data, for example.

In an example application, the user has instrumented the object 1604 to be interacted with, the mat 10 is powered on, and the user and object 1604 is near the embedded antenna 1602. In this example, a user picks up an object 1604 that has been instrumented with a Gen 2 UHF 902-928 MHz RFID (GEN2) tag. The user steps onto a surface of the mat 10 with the object 1604 in hand. While on the surface the object's 1604 relative distance can be determined by the mat 10 using collected input data from the embedded antenna 1602. The user may perform cyclical actions with the object 1604 such as shoulder presses or bicep curls. In real time, metrics can be derived from the relative position of the object 1604 such as repetition and velocity.

The object 1604 can have variable composition, such as metal, plastic, concrete, or a mix of the previous that is instrumented with a GEN2 tag on the surface. The objects distance can be detectable above the surface of the mat 10 (e.g. up to a maximum of 3.6 meters). In this example, the mat 10 can have a surface that is rollable with an embedded RFID reader module and antenna 1602 (e.g. M6E-NANO).

Embodiments described herein can provide an application programming interface (API) for a mat 10, such as a heated yoga mat 10. Content can have embedded instructions for patterns of map output that has the ability to control the input controls 22 and output components 24 on a connected yoga mat 10 in real-time or post-time through a wired or wireless connection to enable a content-controlled experience. The mat 10 also can return the states of each input control 22 back to a device providing the content to enable a feedback loop. Example inputs include temperature, display technology, and pressure.

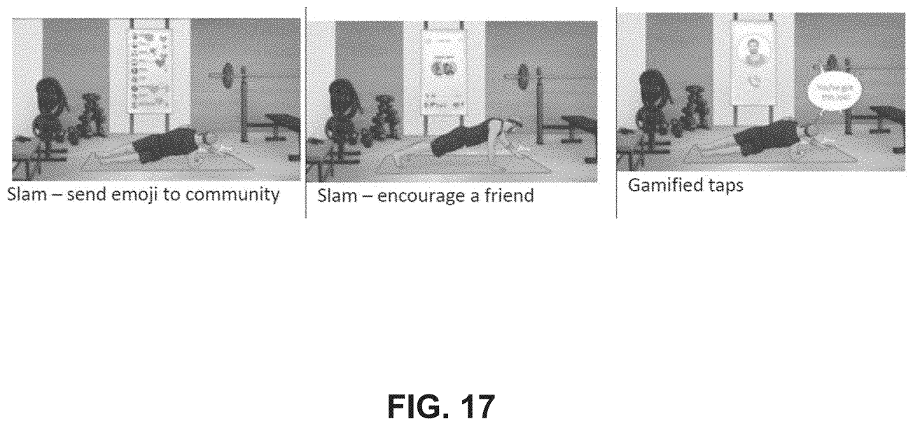

In some embodiments, the mat 10 provides a new way of connecting a community of users. The mat 10 can enable new ways of connecting with others while working out with a remote instructor (via instructor device 40). An input control 22 (e.g. button) on the mat may be a multi-purpose button which changes its purpose or function overtime and throughout a session. That is, the input control 22 can have different states linked to different functions or purposes. FIG. 17 shows an example of different community immersive examples or gestures to connect with other users using the mat 10. For example, in one instance, the input control 22 may be used to virtually “hi-five” another participant. In another instance, a user might press down the input control 22 to instantly speak to another participant and give them some words of encouragement. In another instance, the user may be using the input control 22 as a means to give feedback to the instructor/community (e.g. tap the button to send hearts if you're loving this workout!”).

In some example embodiments, the input control 22 of the mat 10 is a touch sensor. Different technologies can be used to implement an input control 22 (or other input device) on the mat 10. Two example technologies are Force Sensing Resistors (FSR) and tactile sensors for a combination of robustness, performance, cost and manufacturability. For example, an input control 20 can involve using pressure sensors built into the mat 10 to simulate a “button” on the mat 10 (e.g. in a corner of the mat 10). As another example, an input control 20 can be an FSR embedded in the mat 10. As a further example, an input control 20 can be capacitive touch sensors that can enable interactions that require touch or do not require touch. Interactions above the mat 10 in the air, at a given distance can be possible with this sensor.

As another example, an input control 20 can be tactile sensors of mechanical switches designed specifically for the mat 10. For example, two foil leaves can be placed between a foam core or insulated core. As a further example, an input control 20 can be piezoelectric film sensors. The sensors develop a small voltage when the film is stressed. A control system of mat 10 can interpret these signals to trigger different commands and outputs. As another example, an input control 20 can be conductive foams with digital signal processing.

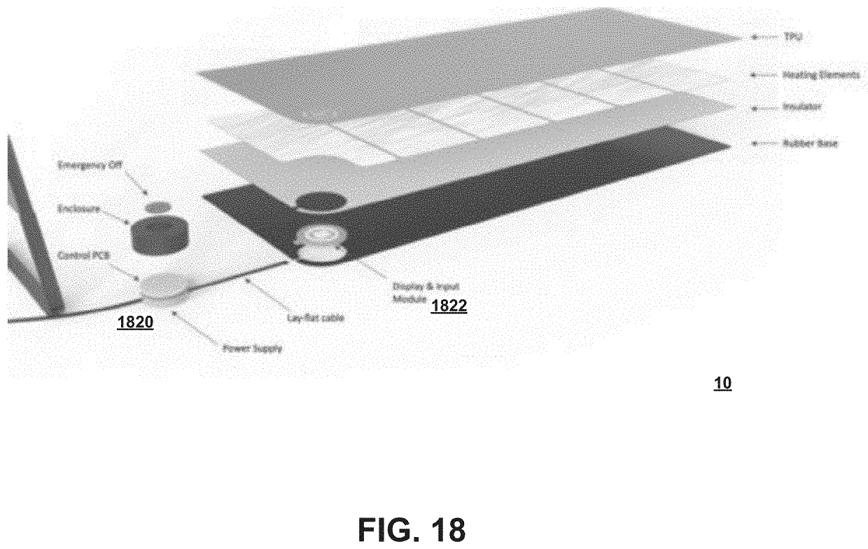

FIG. 18 is another example diagram of an exploded view of the mat 10 to illustrate different layers of the mat 10. In this example, the mat 10 has four different layers: a Tensor Processing Unit (TPU) layer, heated layer with multiple heating elements, an insulator layer, and a rubber base layer. The mat 10 has an integrated display/input component 1822 to receive control commands and selections of patterns of map output, for example. The display/input component 1822 can implement the input control 22 to receive a selected pattern of map output. The display/input component 1822 can also provide visual outputs. The mat 10 also has a control printed circuit board (PCB) 1820 to provide a hardware processor for the mat 10. The control PCB 1820 receives and transmits control commands for mat output, and can also process data. In this example, the control PCB 1820 connects to a power supply to receive power, and also connects to the mat 10 (and its display/input component 1822) by a lay-flat cable. The control PCB 1820 has an enclosure for protection and an emergency off button to turn off the electronics unit.

The mat 10 is designed to direct the diffusion of heat into and towards the person standing on the mat 10 (e.g. the mat 10 directs the diffusion of heat upwards) instead of diffusion of heat into the ground. In this example, the top TPU layer is thin to maximise diffusion of heat. The top TPU layer may also be made of a conductive elastomer, or a material with conductive elements embedded into it to increase the thermal conductivity of the material. For example, small, conductive beads of metal may be embedded in the TPU layer to increase its overall thermal conductivity. Additionally, the direction of diffusion can be facilitated by introducing a thermally insulator layer with a high specific heat capacity underneath the heated layer of heating elements. In this way, the diffusion of heat can be directed towards the materials with lower specific heat capacity and lower thermal insulative properties (e.g. by being thin) on the upper surface of the mat 10.

The mat 10 generates sensory output according to different patterns of map output to create different sensory experiences for the user.

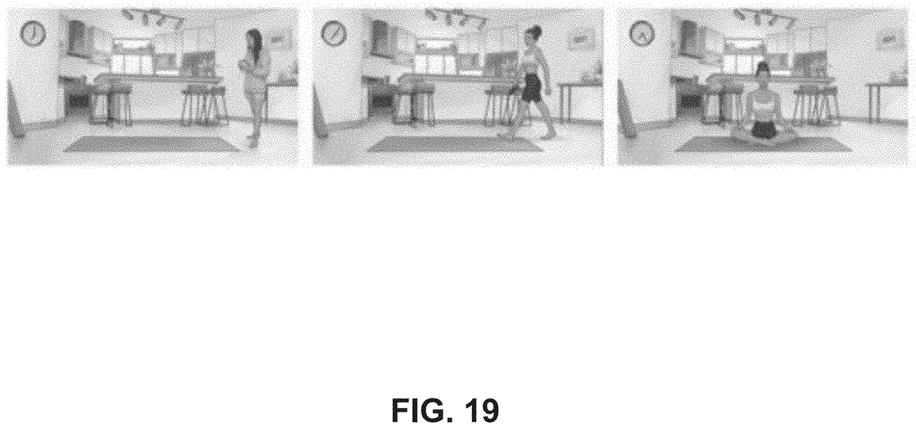

FIG. 19 illustrates example heat experiences generated by mat 10. A heat experience can involve pre-heat experience such that the mat 10 generates heat before exercise or activity. For example, the mat 10 can generate heat (as an example of sensory output) according to a schedule or time trigger to gently introduce guests to their yoga session, reducing the friction to start and prime the mind-body connection for the yoga session.

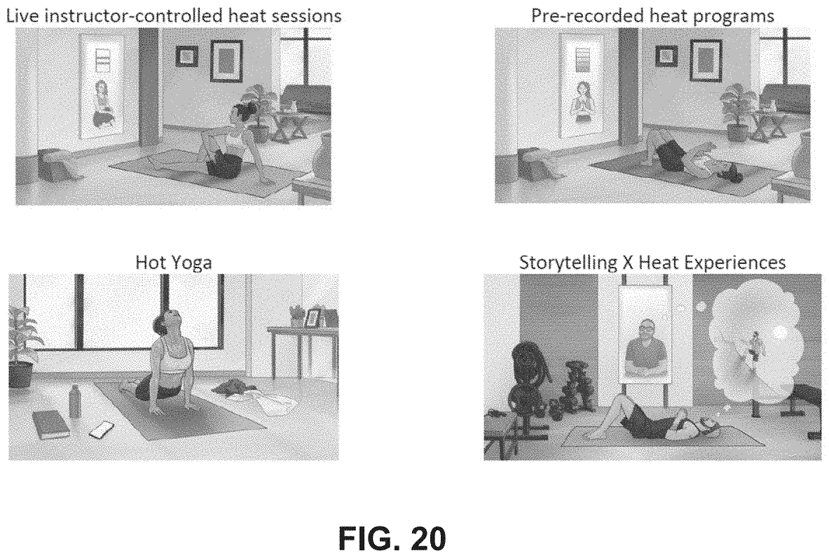

FIG. 20 illustrates further example heat experiences generated by mat 10 for engaging new content. A heat experience can involve the mat 10 synchronizing heat output with content, such as an exercise or activity. For example, the mat 10 can generate heat (as an example of sensory output) according at time intervals that synchronize with time intervals or events of the live content or pre-recorded content to enhance the sensory experience of the content. For example, the mat 10 can generate heat output synchronized at timed intervals with live and pre-recorded content to deepen immersion and increase connection with the instructor during a session. The content can embed instructions for the pattern of map output to synchronize the sensory output with the content. The instructions can be executed by the mat 10 to generate the sensory output. Different examples of content experiences include live instructor-controlled heat sessions, pre-recorded heat programs, hot yoga and storytelling heat experiences.

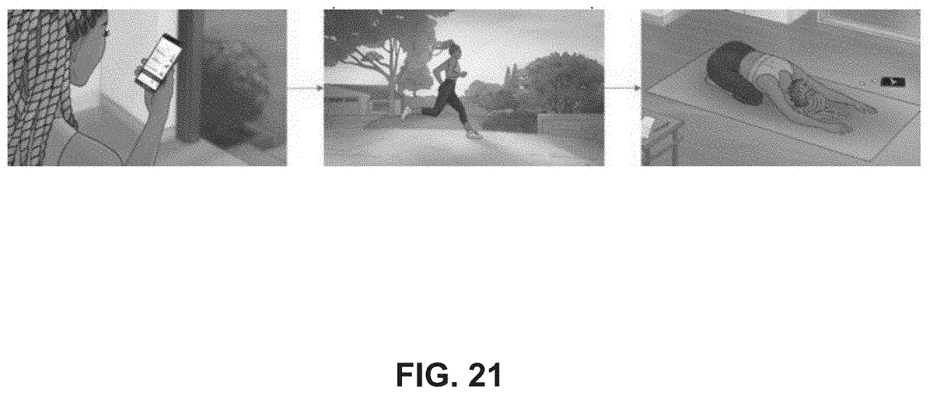

FIG. 21 illustrates further example heat experiences generated by mat 10 for different exercises or activities. In this example, the mat 10 can be used before or after an activity outside to warm up and/or cool down before or after a run or other activity outside.

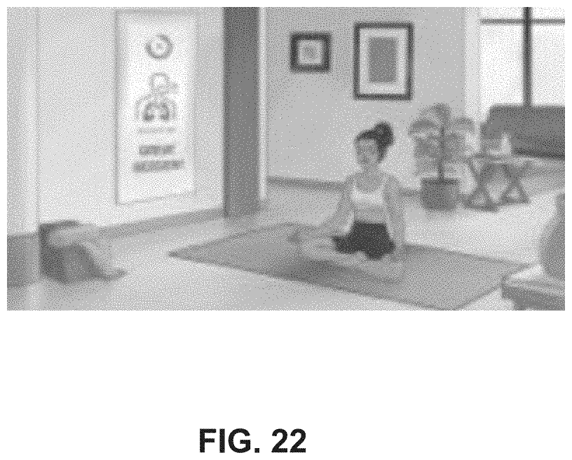

FIG. 22 illustrates further example heat experiences generated by mat 10. In this example, the mat can use heat output as an indicator to signal timing for transitions. Content may involve transitions from one activity to another and the heat output can provide a gentle indicator to signal timing for transitions.

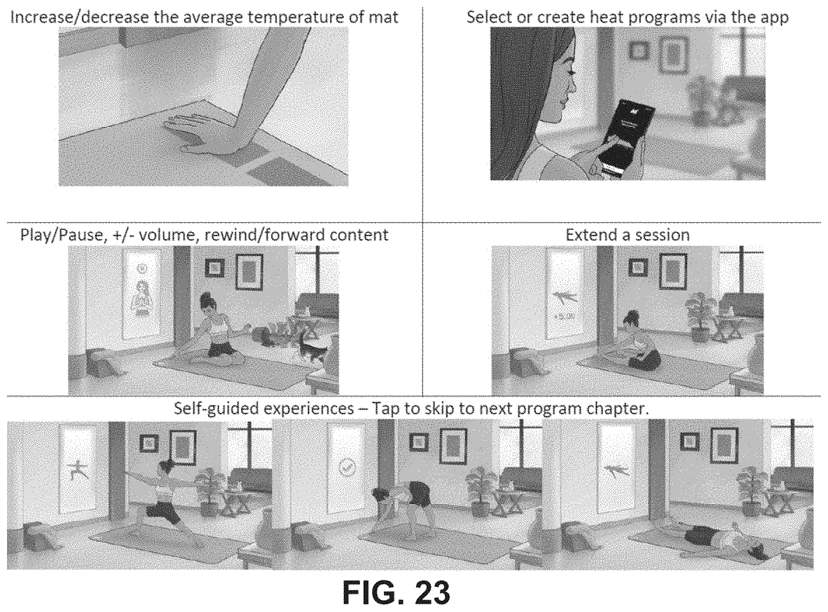

FIG. 23 illustrates example control experiences generated by mat 10. The control experiences can generate different types of control commands to trigger mat output. Example control experiences relate to heat controls to increase or decrease the average temperature of the mat 10, or to select or create heat programs (e.g. patterns of mat output that involve heating elements) using a client application. Example control experiences relate to content controls to manipulate playback of content (e.g. play/pause, increase/decrease volume, rewind/forward), to extend a content session (e.g. increase duration of content), modify or trigger self-guided experiences by tapping mat to skip to next program or chapter.

As previously noted, FIG. 17 illustrates examples of connecting with a community of users or content using controls triggered by touching the mat 10 or by input controls 22 of mat.

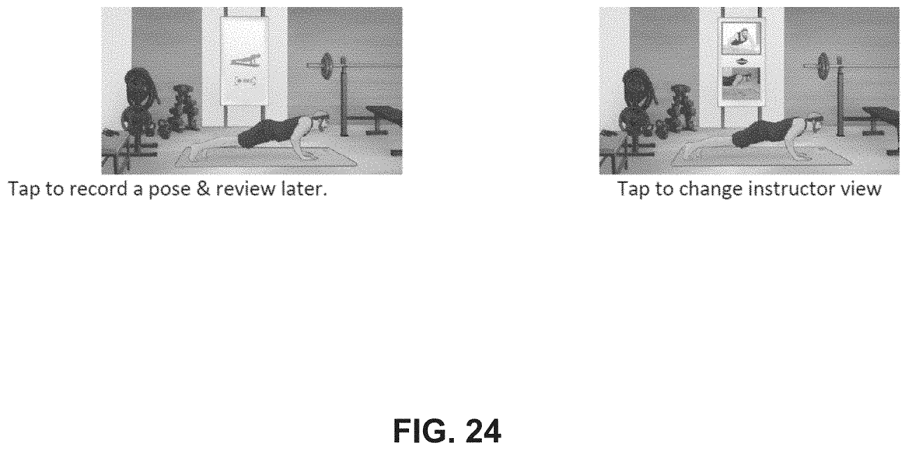

FIG. 24 illustrates example experiences generated by mat 10. For example, an input control 22 can be activated (e.g. touch region of mat 10) to trigger a camera to record a pose and later playback the recording to review the pose. As another example, an input control 22 can be activated (e.g. touch region of mat 10) to trigger a change in a field of view for a camera or an instruction view of the mat 10 (and a user on the mat 10).

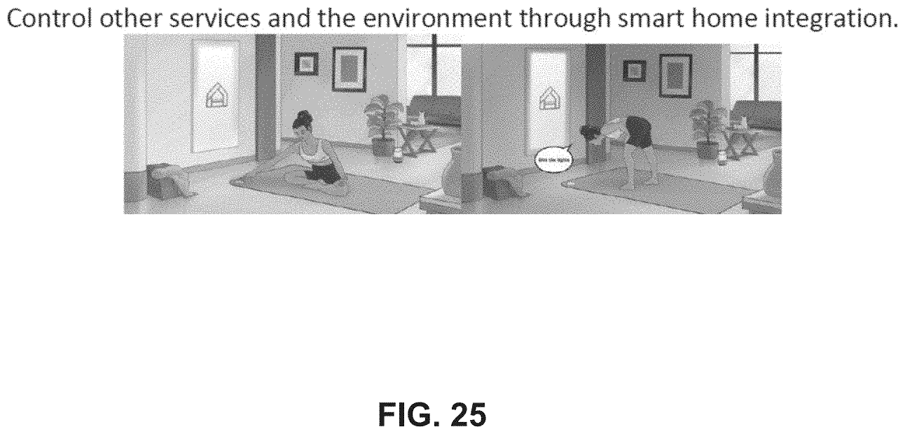

FIG. 25 illustrates example experiences generated by mat 10 for smart home integration. For example, an input control 22 of the mat 10 can be used to control services and environment (e.g. lighting, speakers, temperature) by actuating devices or peripherals to provide smart home integration.

Accordingly, embodiments described herein provide a connected workout mat 10 to create multisensory experiences. The connected mat 10 has at least one input control 22 to receive a selected pattern of mat output from a plurality of patterns of mat output and at least one output component 24 to provide sensory output based on the selected pattern of mat output. The mat 10 can synchronize the sensory output with content from a digital content platform that can be displayed to a user at an immersive hardware device. The connected mat 10 has a connection to a hardware processor that provides instructions for the selected pattern of mat output and the content. The sensory output can involve different peripherals (e.g. speaker 1208, light 1214).

In some embodiments, the hardware processor is removable from the mat 10. In some embodiments, the hardware processor is embedded in the mat 10. In some embodiments, the mat 10 has a plurality of layers such as a non-slip textile surface layer, a multi-zone heating layer, a sensor layer, a mat base layer. In some embodiments, the at least one input device comprises an input surface and a sensor layer integrating a plurality of sensors. In some embodiments, the mat has a light portion that provides an indicator and a touch surface.

In another aspect, embodiments described herein provide a workout mat 10 to create sensory experiences. The connected mat generates visual output using a display device based on the input data.

In some embodiments, the input control comprises a low-power controller integrated into the mat to consolidate signals from sensors of the sensor layer, and direct controls to zones of the multi-zone heating layer, wherein power and data is transmitted to the controller by a connector.

In some embodiments, the input control receives instructor input by a remote instructor device for direct manipulation and control of the sensory output for a workout experience through a range of outputs comprising heating, cooling, and vibration exhibited through the connected mat.

The following discussion provides many example embodiments. Although each embodiment represents a single combination of inventive elements, other examples may include all possible combinations of the disclosed elements. Thus if one embodiment comprises elements A, B, and C, and a second embodiment comprises elements B and D, other remaining combinations of A, B, C, or D, may also be used.

The term “connected” or “coupled to” may include both direct coupling (in which two elements that are coupled to each other contact each other) and indirect coupling (in which at least one additional element is located between the two elements).

The technical solution of embodiments may be in the form of a software product. The software product may be stored in a non-volatile or non-transitory storage medium, which can be a compact disk read-only memory (CD-ROM), a USB flash disk, or a removable hard disk. The software product includes a number of instructions that enable a computer device (personal computer, server, or network device) to execute the methods provided by the embodiments.

Although the embodiments have been described in detail, it should be understood that various changes, substitutions and alterations can be made herein without departing from the scope as defined by the appended claims.

Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed, that perform substantially the same function or achieve substantially the same result as the corresponding embodiments described herein may be utilized. Accordingly, the appended claims are intended to include within their scope such processes, machines, manufacture, compositions of matter, means, methods, or steps.

As can be understood, the examples described above and illustrated are intended to be exemplary only. The scope is indicated by the appended claims.

What is claimed is:

1. A workout mat to enhance digital connection through sensory output of a physical connected workout surface of the mat responsive to input, wherein the workout mat comprises at least one input control to receive a selected pattern of mat output from a plurality of patterns of mat output, and at least one output component to provide sensory output according to the selected pattern of mat output.

2. The workout mat of claim 1 wherein the plurality of patterns of mat output comprise at least one selectable pre-programmed pattern of mat output.

3. The workout mat of claim 1 wherein the plurality of patterns of mat output comprise at least one modifiable pattern of mat output.

4. The workout mat of claim 1 wherein a client application on an electronic device has a user interface to generate a user profile to define one or more attributes of the selected pattern of mat output, wherein the one or more attributes are linked to at least one of a selected exercise and a selected experience.

5. The workout mat of claim 1 wherein the mat is connectable to at least one input device to receive input data to trigger content at a digital content platform, wherein the input control receives instructions for the sensory output from the content, the instructions being embedded within the content.

6. The workout mat of claim 5 wherein the input control receives instructor input from an instructor device to modify the instructions for the sensory output.

7. The workout mat of claim 1 wherein the at least one output component synchronises the sensory output with content at a digital content platform.

8. The workout mat of claim 1 wherein the sensory output of the physical connected workout surface is controlled or activated by exercise content of a digital content platform, wherein the content has one or more timestamps or metadata to synchronize the sensory output with the exercise content.

9. The workout mat of claim 1 comprising a non-slip textile surface layer, a multi-zone heating layer, a sensor layer, a mat layer.

10. The workout mat of claim 9 wherein the input control comprises a low-power controller integrated into the mat to consolidate signals from sensors of the sensor layer, and direct controls to zones of the multi-zone heating layer, wherein power and data is transmitted to the controller by a connector.

11. The workout mat of claim 10 wherein the low-power controller is removable.

12. The workout mat of claim 1 comprising a light portion that provides an indicator and a touch surface.

13. The workout mat of claim 1 wherein the input control comprises an input surface and a sensor layer integrating a plurality of sensors.

14. The workout mat of claim 1 wherein the at least one input control captures interactions with the connected mat to trigger control commands relating to the selected pattern of pattern of mat output, the interactions comprising gesture recognition, taps in zones on the mat, and intelligent activity recognition based on context.

15. A system for a workout mat to create sensory experiences, the system comprising:
a workout mat with at least one input device to receive input data for a pattern of map output and at least one output component to provide a sensory output according to the pattern of map output;
a hardware processor that receives selected exercise content of a digital content platform and provides the pattern of map output to the at least one input control based on the selected exercise content to synchronize the selected exercise content of the digital content platform and the sensory output at the connected mat.

16. The system of claim 15 further comprising a client application on an electronic device with a user interface to generate a user profile to define one or more attributes of the pattern of mat output.

17. The system of claim 15 wherein the hardware processor processes the input data using a trained model,
wherein the hardware processor updates the trained model over time based at least on one dataset where the dataset relates to at least one of user specific data, household specific data, ambient environment data, community data, expert data, instructor data, user goal data, community goal data, instructor goal data, data captured from other users, and content.

18. The system of claim 15 wherein the hardware processor controls content by interactions with the connected mat captured by at least one input control to trigger control commands relating to the pattern of mat output, the interactions comprising gesture recognition, taps in zones on the mat, and intelligent activity recognition based on context.

19. The system of claim 15 further comprising an instructor device that provides control commands to the digital content platform to modify the pattern of map output, and
to control a workout experience through a range of outputs comprising heating, cooling, and vibration exhibited through the connected mat.

20. A method for a connected workout mat to create sensory experiences, the method comprising:
providing a plurality of selectable patterns of mat output;
receiving a selected pattern of mat output from a connected mat having at least one input control; and
generating sensory output by at least one output component of the connected mat based on the selected pattern of map output.
